(19) United States
(12) Patent Application Publication
(10) Pub. No.: US 2024/0075000 A1
(43) Pub. Date: Mar. 7, 2024

(54) TREATMENT OF PATHOLOGICAL FATIGUE WITH OXALOACETATE

(71) Applicant: Alan B. Cash, San Diego, CA (US)

(72) Inventor: Alan B. CASH, San Diego, CA (US)

(21) Appl. No.: 18/272,063

(22) PCT Filed: Jan. 13, 2022
(86) PCT No.: PCT/US2022/012327

§ 371 Date: Jul. 12, 2023

Related U.S. Application Data
(60) Provisional application No. 63/137,524, filed on Jan. 14, 2021.

(51) Int. Cl. A61K 31/194

(52) U.S. Cl. CPC A61K 31/194

(57) ABSTRACT

The present disclosure relates to methods of treatment and compositions for treatment of Pathological Fatigue caused by injury or disease in the body. Pathological fatigue refers to physical and mental fatigue that is caused by viral infection, bacterial infection, trauma, disease, or genetic alteration that results in fatigue that is not improved by bed rest and may be worsened by physical or mental activity. Such pathologic fatigue occurs in myalgic encephalomyelitis (ME)/Chronic Fatigue Syndrome (CFS) and other disorders such as such as post-COVID-19 fatigue, post-viral fatigue, fibromyalgia (FM), cancer, Parkinson's Disease, other diseases and trauma as well as of combinations thereof. Related treatment methods and pharmaceutical compositions are also disclosed.

### CROSS REFERENCE TO RELATED APPLICATIONS

This application claims the benefit of U.S. Provisional Application No. 63/137,524, filed on Jan. 14, 2021, the entire contents of which are incorporated herein by reference.

### FIELD OF THE INVENTION

The present disclosure relates to methods of treatment and compositions for treatment of Pathological Fatigue caused by injury or disease in the body. Pathological fatigue refers to physical and mental fatigue that is caused by viral infection, bacterial infection, trauma, disease, or genetic alteration that results in fatigue that is not improved by bed rest and may be worsened by physical or mental activity. Such pathologic fatigue occurs in myalgic encephalomyelitis (ME)/Chronic Fatigue Syndrome (CFS) and other disorders such as such as post-COVID-19 fatigue, post-viral fatigue, fibromyalgia (FM), cancer, Amyotrophic Lateral Sclerosis (ALS), Parkinson's disease, other diseases and trauma as well as of combinations thereof. Related treatment methods and pharmaceutical compositions are also disclosed.

### BACKGROUND OF THE INVENTION

Physiological Fatigue is familiar to most persons, primarily resulting from exertion, that is, an inability to continue exercise at the same intensity with a resultant deterioration in performance (Evans W J, Lambert C P. Physiological basis of fatigue. Am J Phys Med Rehabil. 2007; 86(1 Suppl):S29-46.) It can also be caused by sleep loss or extended wakefulness, disrupted circadian rhythm or increased workload (Lock A M, Bonetti D L, Campbell A D K. The psychological and physiological health effects of fatigue. Occup Med (Lond). 2018; 68(8):502-11). In contrast, Pathological fatigue or pathological exhaustion is more than tiredness (Barnett R. Fatigue. Lancet. 2005; 366(9479):21) and refers to physical and mental fatigue that is caused by viral infection, bacterial infection, trauma, disease, over-work, over-training, or genetic alteration that results in physical and mental fatigue that is not improved by bed rest and may be worsened by physical or mental activity.

Physiological Fatigue is caused by neurological changes, calcium level changes, blood flow and oxygen levels, reduced ATP energy levels and glycogen levels, and an increase in intracellular metabolites such as H+, lactate, Pi and ROS as summarized in Wan, et. al. (2017) (Wan J J, Qin Z, Wang P Y, Sun Y, Liu X. Muscle fatigue: general understanding and treatment. Exp Mol Med. 2017; 49(10):e384). Most importantly, these physiological changes are reversed by rest.

In contrast, while Pathological Fatigue may involve some of the same physiological changes seen in physiological fatigue, there are many additional metabolic changes that occur in Pathological Fatigue, including in energy production pathways, redox of the cells, inflammation response, mitochondrial malfunction, and reduced AMPK activation (and related glucose tissue uptake). Unlike Physiological Fatigue, the metabolic changes in Pathological Fatigue are not reversed by rest, and the fatigue may last long after the virus has been conquered, the bacterial invasion has been defeated, or the damaged tissue repaired.

Specific examples of this include the disorder Myalgic encephalomyelitis/Chronic Fatigue Syndrome (ME/CFS). Because ME/CFS can be triggered by many different insults to the body, the disorder is based on on-going symptoms rather than the cause of the disorder. The disorder is characterized by persistent fatigue and other specific symptoms that last for a minimum of six months in adults (and 3 months in children or adolescents), and the fatigue is not relieved by bed rest. This disease is also referred to as systemic exertion intolerance disease (SEID), post-viral fatigue syndrome (PVFS) and chronic fatigue immune dysfunction syndrome (CFIDS).

ME/CFS patients have persistent and debilitating fatigue, diffuse musculoskeletal pain, sleep disturbances, neuropsychiatric symptoms and cognitive impairment such as brain fog which cannot be explained by an underlying medical condition. The symptoms of ME/CFS are not caused by ongoing exertion and are not relieved by rest.

ME/CFS is a symptom-based diagnosis or clinical diagnosis without distinguishing physical examination or routine laboratory findings. Infectious, immunological, neuroendocrine, sleep and psychiatric mechanisms have been investigated; however, a unifying etiology for ME/CFS has not yet emerged. The majority of ME/CFS cases start suddenly and they are usually accompanied by a “flu-like illness”, while a significant proportion of cases begin within several months of severe adverse stress (Afari N et al (2003), Am J Psychiatr 160 (2): 221-36). Often, there are courses of remission and relapse of symptoms which make the illness difficult to manage. Persons who feel better for a period may overextend their activities, and the result can be a worsening of their symptoms with a relapse of the illness.

Viral infections have been causally linked to cases of ME/CFS. 40% of the people infected with the corona virus SARS had chronic fatigue after infection, and 27% of these patients met the Center for Disease Control's criteria for ME/CFS. 11% of the people diagnosed with the Ross River Virus, Epstein-Barr Virus or Q-fever virus were diagnosed with ME/CFS six months later. (Hickie I, Davenport T, Wakefield D, Vollmer-Conna U, Cameron B, Vernon S D, et al. Post-infective and chronic fatigue syndromes precipitated by viral and non-viral pathogens: prospective cohort study. BMJ. 2006; 333(7568):575). There is great concern that the current COVID-19 pandemic will lead many patients to ME/CFS (Perrin R, Riste L, Hann M, Walther A, Mukherjee A, Heald A. Into the looking glass: Post-viral syndrome post COVID-19. Med Hypotheses. 2020; 144:110055.) (7), and indeed, pathological fatigue is one of the most common symptoms of COVID-19, in one study affecting 55% of the patient population. (Jacobs L G, Gourna Paleoudis E, Lesky-Di Bari D, Nyirenda T, Friedman T, Gupta A, et al. Persistence of symptoms and quality of life at 35 days after hospitalization for COVID-19 infection. PLoS One. 2020; 15(12):e0243882.) (8)

Cancer and cancer treatment often cause lasting fatigue, even when the patients are in remission from the disease. Prevalence rates of pathological fatigue range from 59 to nearly 100% depending on the clinical status of the cancer. (Weis J. Cancer-related fatigue: prevalence, assessment and treatment strategies. Expert Rev Pharmacoecon Outcomes Res. 2011; 11(4):441-6). Cancer related fatigue can be more distressing and longer lasting than the disease itself. The fatigue persists even though the cancer is no longer present, and the patients are not taking chemotherapy.

ME/CFS often occurs together with other diseases such as fibromyalgia (FM), multiple chemical sensitivities, irritable bowel syndrome and temporomandibular joint disorder. In particular, co-morbidity with fibromyalgia has been studied (Afari N et al, supra). Fibromyalgia is a non-articular rheumatic syndrome characterized by myalgia and multiple points of focal muscle tenderness to palpation (trigger points). Patients with FM often experience muscle pain aggravated by inactivity or exposure to cold. This condition is often associated with general symptoms, such as sleep disturbances, fatigue, stiffness, headaches, anxiety, perceived stress and occasionally depression.

Despite the contrasting definitions, 20-70% of patients with fibromyalgia also meet the criteria for chronic fatigue syndrome, and conversely, 35-70% of those with chronic fatigue syndrome-like illnesses have concurrent fibromyalgia (Afari N et al, supra).

ME/CFS is a common disorder. Estimates of the prevalence of ME/CFS range from 0.07% to 2.8% in the general adult population and is lower in children and adolescents (Afari N et al, supra). The prevalence of the related fibromyalgia (FM) is 2-4%. This means that in the United States at least 230,000 patients suffer from ME/CFS and 6.5 million from FM (for review see Zachrisson O (2002); Fatigue Syndrome-aspects on biology, treatment and symptom evaluation. ISBN 91-628-5386-4. Gothenburg University).

Many patients suffering from ME/CFS experience significant functional impairment. Nearly all patients with ME/CFS notice a decrease in social relationships in addition to other unwanted consequences of illness; about one-third are unable to work or study, and another one-third can only work part-time (Afari N et al, supra). Many patients suffering from ME/CFS also experience depression symptoms and are diagnosed with clinical depression, and likewise, patients who suffer from depression often experience symptoms of debilitating fatigue.

Currently, patients suffering from ME/CFS are treated by cognitive behavioral therapy (CBT) or graded exercise therapy (GET), which have shown moderate effectiveness in multiple randomized controlled trials, however many patients do not make recovery (Rimes K A et al (2005), (10) Occupational Medicine 55(1): 32-39; Chambers D et al (2006). Journal of the Royal Society of Medicine 99(10): 506-20). At present, medication plays a minor role in disease management (Van Houdenhove B et al (2010) Expert opinion on pharmacotherapy 11(2): 215-23).

Additionally, many disorders, in addition to ME/CFS and FM, are characterized by symptoms of debilitating fatigue. Such disorders include post-COVID-19 fatigue, post-viral fatigue, post-bacterial infection fatigue, mental fatigue, post stroke fatigue, Huntington's disease, Parkinson's disease, Amyotrophic Lateral Sclerosis (ALS), multiple sclerosis, narcolepsy, post cancer fatigue, fatigue associated with cancer with or without cytostatic treatment, depression, and combinations thereof. None of these fatigues stem from over-exertion of muscle tissues, and rest does not provide a cure for this type of fatigue.

Muscle fatigue is easily cured by rest, allowing the nutrients to be taken in by the muscles and waste products such as lactate to be removed by normal cellular processes. In fatigue due to damage, whether from viral or bacterial infection, trauma, disease or other cellular assault, cellular metabolism changes do not always re-set after providing energy for the defense/repair of the body. The failure of metabolism to re-set back to a normal state leads to on-going mental and physical fatigue, which can be lasting for years, even after the original insult to the body is resolved.

Various metabolic mechanisms are turned on the by damage to the body, and these ongoing metabolic changes can cause lasting fatigue if they are not reprogramed back to the original normal metabolic state. One such metabolic change is the increase in glycolysis in the cytoplasm of the cell. Using an ancient metabolic feature, cells can increase their consumption of glucose, ferment the glucose in the cytosol to lactate to increase energy production over the energy produced by the mitochondrial alone. Typically, in non-stressed healthy cells, the mitochondria burn glucose through oxidative phosphorylation rather than ferment glucose in the cytosol. Burning glucose in the mitochondria is much more efficient and creates less toxic end-products than fermentation in the cytosol. However, stressed, unhealthy cells often turn to increased glycolysis/fermentation for extra energy in times of perceived need. This shift in metabolism was first described by Otto Warburg in the 1930's and has been named the “Warburg Effect”. Warburg described the metabolic energy shift in relation to cancer cells, and indeed, almost all cancers exhibit this change in energy metabolism. Otto Warburg though that once the cell changed to this different energy production method, that it could not change back into a normal cell. This energy pathway change can lead to pathological fatigue. (Warburg O. On the origin of cancer cells. Science. 1956; 123(3191):309-14).

The Warburg Effect is not only present in cancer cells but is seen in adaptive immune cells of myeloid and lymphoid lineage, characterized by a shift to aerobic glycolysis. (Kornberg M D. The immunologic Warburg effect: Evidence and therapeutic opportunities in autoimmunity. Wiley Interdiscip Rev Syst Biol Med. 2020; 12(5):e1486). The Warburg Effect is present in the replication of viruses such as MERS-CoV and SARS-CoV-2. (Icard P, Lincet H, Wu Z, Coquerel A, Forgez P, Alifano M, et al. The key role of Warburg effect in SARS-CoV-2 replication and associated inflammatory response. Biochimie. 2020; 180:169-77. Clinical work in ME/CFS patients shows this change to Warburg Effect metabolism, generating most of the energy currency ATP from non-mitochondrial sources. (Lawson N, Hsieh C H, March D, Wang X. Elevated Energy Production in Chronic Fatigue Syndrome Patients. J Nat Sci. 2016; 2(10)).

Another metabolic change seen in fatigued patients is the decrease in the ratio of pyridine nucleotides nicotinamide adenine dinucleotide (NAD+) to its reduced state NADH in the cytoplasm, also known as a change in the “Redox” of the cell. NAD+ levels in the cell act as a signaling molecule to drive certain metabolic states. A review of the importance of NAD+ levels to metabolism and skeletal muscle function is reviewed in White et. al. (2012). (White A T, Schenk S. NAD(+)/NADH and skeletal muscle mitochondrial adaptations to exercise. Am J Physiol Endocrinol Metab. 2012; 303(3):E308-21). In humans, NAD+ levels decrease with muscle use. As an example of this, Graham et. al (1978) found that muscle NAD+ levels are decreased with exercise at 65% and 100% of maximal oxygen uptake (VO₂ max), and although increased muscle water accounted for ˜73% of this decrease, NAD+ levels were still reduced when assessed on a dry weight basis (Graham T, Sjogaard G, Lollgen H, Saltin B. NAD in muscle of man at rest and during exercise. Pflugers Arch. 1978; 376(1):35-9). NADH levels also increase (Sahlin K, Katz A, Henriksson J. Redox state and lactate accumulation in human skeletal muscle during dynamic exercise. Biochem J. 1987; 245(2):551-6), which further drives down the NAD+/NADH ratio. Sweetman et. al. (2020) calculated that NADH levels are higher in peripheral blood mononuclear cells in patients with ME/CFS (Sweetman E, Kleffmann T, Edgar C, de Lange M, Vallings R, Tate W. A SWATH-MS analysis of Myalgic Encephalomyelitis/Chronic Fatigue Syndrome peripheral blood mononuclear cell proteomes reveals mitochondrial dysfunction. J Transl Med. 2020; 18(1):365).

Yet another metabolic change that takes place in response to cellular stress/damage is the translocation of the protein complex Nuclear Factor Kappa-light-chain-enhancer of activated B cells (NF-κB) from the cytoplasm to the nuclear compartment. This translocation allows interaction with the chromatin and the production of inflammation proteins for tissue defense/repair. While this response is critical for keeping us healthy, in some persons the response does not shut-off, such as in COVID-19 patients with Long-Haul symptoms, and the energy of the cell is continually tied up in immune response (Afrin L B, Weinstock L B, Molderings G J. Covid-19 hyperinflammation and post-Covid-19 illness may be rooted in mast cell activation syndrome. Int J Infect Dis. 2020; 100:327-32). This inflammation pathway change can lead to on-going fatigue and is seen in the diseases that have fatigue as a common determinant (Gupta S C, Kim J H, Kannappan R, Reuter S, Dougherty P M, Aggarwal B B. Role of nuclear factor kappaB-mediated inflammatory pathways in cancer-related symptoms and their regulation by nutritional agents. Exp Biol Med (Maywood). 2011; 236(6):658-71; Bower J E, Ganz P A, Irwin M R, Arevalo J M, Cole S W. Fatigue and gene expression in human leukocytes: increased NF-kappaB and decreased glucocorticoid signaling in breast cancer survivors with persistent fatigue. Brain Behav Immun. 2011; 25(1):147-50; Morris G, Maes M. Increased nuclear factor-kappaB and loss of p53 are key mechanisms in Myalgic Encephalomyelitis/chronic fatigue syndrome (ME/CFS). Med Hypotheses. 2012; 79(5):607-13).

Mitochondria are organelles that produce most of the energy during normal cell function. Increased energy demands to fight infection and repair tissues can increase the production of reactive oxygen species (ROS) within the mitochondria, damaging mitochondrial function. Mitochondrial malfunction is implicated in fatigued patients (Filler K, Lyon D, Bennett J, McCain N, Elswick R, Lukkahatai N, et al. Association of Mitochondrial Dysfunction and Fatigue: A Review of the Literature. BBA Clin. 2014; 1:12-23).

Yet another metabolic change that takes place in response to cellular stress/damage is reduced activation of the AMPK protein, and a resulting reduction in glucose uptake by tissues. This is seen directly in cells from ME/CFS patients (Brown A E, Jones D E, Walker M, Newton J L. Abnormalities of AMPK activation and glucose uptake in cultured skeletal muscle cells from individuals with chronic fatigue syndrome. PLoS One. 2015; 10(4):e0122982). Reductions in the fuel available to the cell can be a direct source of fatigue.

Options for treatment of fatigue are extremely limited. No pharmaceutical is approved for the treatment of fatigue. Modafinil, methylphenidate, amantadine, amphetamine and dextroamphetamine have been used in an “off-label” fashion. Each of these drugs has significant side effects. For example, for each of these drugs, animal reproduction studies have shown an adverse effect on the fetus and there are no adequate and well controlled studies in humans for fatigue (world wide web at drugs.com/condition/fatigue.html Jan. 5, 2020)

Caffeine has often been touted for improving cognitive attention, (Repantis D, Bovy L, Ohla K, Kuhn S, Dresler M. Cognitive enhancement effects of stimulants: a randomized controlled trial testing methylphenidate, modafinil, and caffeine. Psychopharmacology (Berl). 2020; Herden L, Weissert R. The Effect of Coffee and Caffeine Consumption on Patients with Multiple Sclerosis-Related Fatigue. Nutrients. 2020; 12(8)) but has minimal effects on muscular performance (Harty P S, Stratton M T, Escalante G, Rodriguez C, Dellinger J R, Williams A D, et al. Effects of Bang® Keto Coffee Energy Drink on Metabolism and Exercise Performance in Resistance-Trained Adults: A Randomized, Double-blind, Placebo-controlled, Crossover Study. J Int Soc Sports Nutr. 2020; 17(1):45). A meta-study review of caffeine reported an improvement in endurance in bench-press exercise, but contradictory effects in the lower body (Ferreira T T, da Silva J V F, Bueno N B. Effects of caffeine supplementation on muscle endurance, maximum strength, and perceived exertion in adults submitted to strength training: a systematic review and meta-analyses. Crit Rev Food Sci Nutr. 2020:1-14).

Oxaloacetate, a human energy metabolite, has been shown to increase muscle endurance and reduce muscle fatigue in normal cells that have fatigue stimulated by muscle overuse via electrical current applied to the muscle, (Nogueira L. Acute Oxaloacetate Exposure Enhances Resistance to Fatigue in in vitro Mouse Soleus Muscle. FASEB Journal. 2011; 25(1104.5)), but there is no indication that oxaloacetate may be used to treat pathological fatigue due to viral infection, bacterial infection, disease, or trauma, as the fatigue from those conditions are different than muscular fatigue seen from simple muscle overuse.

Thus, there is a large need for novel therapies and treatments to alleviate physical and mental pathological fatigue symptoms after tissue damage from viral infection, bacterial infection, trauma, cancer, and other diseases, and thus the provision thereof remains a matter of substantial interest within the field.

Uses of oxaloacetate are disclosed in U.S. Pat. Nos. 9,050,306, 9,561,199, 10,016,385, 10,137,099 and US Patent Application Publication No. 20190321315A1, all incorporated herein by reference.

All references cited herein, including patent applications and publications, are incorporated by reference in their entirety.

### BRIEF SUMMARY OF THE INVENTION

In some aspects, the invention provides a method of treating one or more symptoms of a disorder characterized by debilitating fatigue in a subject, the method comprising administering a therapeutic amount of an oxaloacetate compound to the subject; wherein said disorder is selected from the group consisting of post-COVID-19 fatigue, post-viral fatigue, myalgic encephalomyelitis/chronic fatigue syndrome, fibromyalgia, mental fatigue, post-stroke fatigue, Amyotrophic Lateral Sclerosis, Myasthenia Gravis, Huntington's disease, debilitating fatigue associated with Parkinson's disease, debilitating fatigue associated with Alzheimer's Disease, multiple sclerosis, narcolepsy, post cancer fatigue, fatigue associated with cancer with or without cytostatic treatment. In some embodiments, said oxaloacetate compound is an anhydrous enol-oxaloacetate. In some embodiments, said oxaloacetate is comprised from the group of enol-oxaloacetate, keto-oxaloacetate, hydrated oxaloacetate, or an oxaloacetate salt. In some embodiments, said disorder is selected from the group consisting of fibromyalgia, mental fatigue, Myalgic encephalomyelitis/chronic fatigue syndrome, fibromyalgia, Huntington's disease, post-COVID-19 fatigue, post-viral fatigue, Amyotrophic Lateral Sclerosis, Myasthenia Gravis, Parkinson's disease, Amyotrophic Lateral Sclerosis (ALS), Alzheimer's disease, multiple sclerosis, and post-cancer fatigue. In some embodiments, said oxaloacetate agent is administered in a dose of approximately 100 to 6,000 mg. In some embodiments, said oxaloacetate agent is administered in a dose of 200 mg to 3,000 mg. In some embodiments, said oxaloacetate agent is administered once, twice or three times a day. In some embodiments, the oxaloacetate compound is in a pharmaceutical composition.

In some aspects, the invention provides a method of treating one or more symptoms of a disorder characterized by debilitating fatigue in a subject, the methods comprising administering a therapeutic amount of a compound to reverse metabolic dysfunction to the subject; wherein said dysfunction is selected from the group consisting of increased glycolysis, chronic activation of NF-kB, decreased NAD+/NADH ratio, mitochondrial insufficiency, reduced activation of AMPK, and combinations thereof. In some embodiments, said compound is an oxaloacetate compound. In some embodiments, said oxaloacetate compound is selected from the group consisting of enol-oxaloacetate, keto-oxaloacetate, hydrated oxaloacetate and an oxaloacetate salt. In some embodiments, said oxaloacetate compound is anhydrous enol oxaloacetate. In some embodiments, said disorder is selected from the group consisting of fibromyalgia, mental fatigue, Myalgic encephalomyelitis/chronic fatigue syndrome, fibromyalgia, Huntington's disease, post-COVID-19 fatigue, post-viral fatigue, Amyotrophic Lateral Sclerosis, Myasthenia Gravis, debilitating fatigue associated with Parkinson's disease, debilitating fatigue associated with Alzheimer's disease, multiple sclerosis, and post-cancer fatigue. In some embodiments, said oxaloacetate compound is administered in a dose of about 100 to about 6,000 mg. In some embodiments, said oxaloacetate compound is administered in a dose of about 200 mg to about 3,000 mg. In some embodiments, said oxaloacetate compound is administered once, twice or three times a day. In some embodiments, the compound to reverse metabolic dysfunction is in a pharmaceutical composition.

### BRIEF DESCRIPTION OF THE DRAWINGS

FIG. 1 shows the results of treatment of COVID-19 fatigue with oxaloacetate. Statistical analysis was by Student's T Test.

FIG. 2 shows the results of treatment of Myalgic Encephalomyelitis/Chronic Fatigue Syndrome (ME/CFS) with oxaloacetate. Statistical analysis was by Student's T Test.

### DETAILED DESCRIPTION OF THE INVENTION

It is an object of the present disclosure to provide to a new and efficacious treatment for patients who are suffering from disorders characterized by persistent and debilitating pathological fatigue.

It is an object of the present disclosure to provide a medicament for use in the treatment of said patients.

It is another object of the present disclosure to provide a method of treatment of a disorder characterized by debilitating pathological fatigue for patients in need thereof.

These and other objects which are evident to the skilled person from the present disclosure are met by different aspects of the invention as claimed in the appended claims and as generally disclosed herein.

The present inventor has unexpectedly found that the clinical outcome in treatment of disorders characterized by debilitating pathological fatigue is significantly improved by reversing multiple metabolic pathways back towards normal (pre-insult) functioning. Thus, as a method of treatment, strategies, compounds or combinations of compounds that modify metabolism in the following pathways will also treat pathological fatigue.
1) Aberrant Energy Production amelioration via Reversal of the “Warburg Effect”, reducing glycolysis and reducing the fermentation production of lactate in the cytoplasm of the cell.
2) Increase in the NAD+/NADH levels in the cell, allowing NAD+ to be more available so that cytoplastic lactate can be converted back to pyruvate. NAD+ levels are the rate limiting portion of the reaction:
   Lactate plus NAD+ converts to Pyruvate plus NADH in the presence of the enzyme lactate dehydrogenase
3) Reduction in the translocation of the protein nuclear factor kappa-light-chain-enhancer of activated B cells (NF-kB) from the cytosol to the nucleus, to stop or reduce inflammatory response and the production of cytokine storms.
4) Increased mitochondrial biogenesis, as higher mitochondrial density can provide higher abilities to process incoming glucose and replace mitochondria that are damaged.
5) Activation of 5′adenosine monophosphate-activated protein kinase (AMPK), an enzyme involved in energy homeostasis, which activates glucose and fatty acid uptake and oxidation when cellular energy is low. Increased glucose uptake provides additional fuel for the cell.

The method of treatment may include one or more of the identified modifications to metabolism to ameliorate pathological fatigue. Strategies, compounds or combinations of compounds to achieve this method of treatment including the following:

The energy metabolite “Oxaloacetate”, which can be a single compound, can be used to modify metabolism in all the above listed metabolic modifications. This metabolite can be in several forms: anhydrous enol-oxaloacetate, enol-oxaloacetate in solution, keto-oxaloacetate in solution, and hydrated oxaloacetate in solution, and combinations thereof. The oxaloacetate can also be part of a salt, such as Sodium Oxaloacetate, or Magnesium Oxaloacetate. Enol-oxaloacetate is also known as hydroxy fumarate. Other names for oxaloacetate include oxaloacetic acid, 2-Oxosuccinic acid, ketosuccinic acid, oxosuccinic acid, 2-ketosuccinic acid, Butanedioic acid, oxo-, Oxaloacetic acetic, Butanedioic acid, 2-oxo-, 2-oxo-butanedioic acid, Oxaloethanoic acid, NSC 77688, UNII-2F399MM81J, alpha-Ketosuccinic acid, EINECS 206-329-8, MFCD00002592, OAA, CHEBI:30744; 2F399MM81J, 2-Ketosuccinate, Ketosuccinate, Oxaloaethanoate, a-Ketosuccinate, 2-Oxosuccinate, 4cts, alpha-Ketosuccinate, a-Ketosuccinic acid, 3-carboxy-3-oxopropanoic acid, oxaloacetic acid, oxaloacetate, oxobutanedioate, 2-oxobutanedioate, 2-oxobutanedioic Acid, oxaloacetate(2-), oxosuccinate, keto-oxaloacetate, 149-63-3, Butanedioic acid, oxo-, ion(2-), oxaoacetate dianion, oxobutanedioic acid, ion(2-), as per the National Library of Medicine, National Center for Biotechnology Information “PubChem” world wide web at pubchem.ncbi.nlm.nih.gov/compound/Oxalacetate#section=Depositor-Supplied-Synonyms reviewed on Jan. 3, 2021.

Oxaloacetate supplementation affects the following dysfunctional metabolic pathways that can be treated by metabolic treatments identified above for pathological fatigue improvement. Specifically, these dysfunctional metabolic pathways are outlined below:
1) Aberrant Energy Production via increased glycolysis in the “Warburg Effect”. Cells from persons with disabling fatigue show aberrant energy production, wherein energy is produced within the cytoplasm via increased glycolysis and fermentation. Oxaloacetate has been shown to reverse this trend, reducing both glycolysis and the formation of lactate. The inventor's efforts to reverse aberrant energy production has been shown even in cancer cells (Ijare O, Conway D, Cash A, Baskin D, Pichumani K. CBMT-49. OXALOACETATE ALTERS GLUCOSE METABOLISM IN GLIOBLASTOMA: 13C ISOTOPOMER STUDY. Neuro-Oncology. 2019; 21(Supplement_6):vi43-vi4) but the effect on fatigue was not evident nor suggested from the cellular tests, and represents a surprising novel finding.
2) Cells from patients with fatigue show significantly lower NAD+/NADH levels. Oxaloacetate increases the NAD+/NADH ratio. (Wilkins H M, Harris J L, Carl S M, E L, Lu J, Eva Selfridge J, et al. Oxaloacetate activates brain mitochondrial biogenesis, enhances the insulin pathway, reduces inflammation and stimulates neurogenesis. Hum Mol Genet. 2014; 23(24):6528-41). The inventor's efforts to reverse the NAD+/NADH ratio has been shown in pre-clinical work (Williams D S, Cash A, Hamadani L, Diemer T. Oxaloacetate supplementation increases lifespan in Caenorhabditis elegans through an AMPK/FOXO-dependent pathway. Aging Cell. 2009; 8(6):765-8) but the effect on fatigue was not evident nor suggested from the pre-clinical tests. Other literature indicates that oxaloacetate supplementation both increases NAD+ and decreases NADH, but the effect on fatigue was not evident, nor suggested in the literature, and represents a surprising novel finding.
3) NF-kB Inflammation Reduction. Cells from persons with disabling fatigue show increased activation of NF-kB which leads to a “cytokine storm”. Oxaloacetate has been shown to reduce the activation of NF-kB by up to 70% in animal models. (Wilkins H M, Harris J L, Carl S M, E L, Lu J, Eva Selfridge J, et al. Oxaloacetate activates brain mitochondrial biogenesis, enhances the insulin pathway, reduces inflammation and stimulates neurogenesis. Hum Mol Genet. 2014; 23(24):6528-41). Although the reduction in the NF-kB pathway by oxaloacetate supplementation was discovered, the effect on fatigue was not evident nor suggested from the animal tests. 4) Mitochondrial damage is prevalent in fatigued patients. Oxaloacetate turns on biomolecular pathways that lead to increased mitochondrial production and density. (Wilkins H M, Koppel S, Carl S M, Ramanujan S, Weidling I, Michaelis M L, et al. Oxaloacetate Enhances Neuronal Cell Bioenergetic Fluxes and Infrastructure. J Neurochem. 2016). The effect of this mitochondrial increase on fatigue was not foreseen, nor suggested in the prior art.
5) AMPK Activation Reduction. Cells from persons with disabling fatigue show an impaired ability to activate AMPK, and impaired stimulation of glucose uptake. Oxaloacetate has been shown to increase glucose uptake in trials of diabetic patients and Alzheimer's patients (Yoshikawa K. Studies on the anti-diabetic effect of sodium oxaloacetate. Tohoku J Exp Med. 1968; 96(2):127-41; Vidoni E D, Choi I Y, Lee P, Reed G, Zhang N, Pleen J, et al. Safety and target engagement profile of two oxaloacetate doses in Alzheimer's patients. Alzheimers Dement. 2020), but the effect on fatigue was not evident from the clinical trials. Additionally, work done in diabetes patients would not be used to treat ME/CFS patients or other fatigue patients.

The “Warburg Effect” refers to a form of modified cellular metabolism, often found in cancer cells, but also found in other cells, which tend to use specialized fermentation of pyruvate to lactate in the cytoplasm over the aerobic respiration pathway that burns pyruvate in the mitochondria that is used by most cells in the body under non-pathological conditions. While fermentation does not produce adenosine triphosphate (ATP) in high yield compared to the citric acid cycle and oxidative phosphorylation of aerobic respiration, it converts nutrients such as glucose and glutamine more efficiently into biomass by avoiding unnecessary catabolic oxidation of such nutrients into carbon dioxide, preserving carbon-carbon bonds and promoting anabolism. (Vander Heiden M G, Cantley L C, Thompson C B. Understanding the Warburg effect: the metabolic requirements of cell proliferation. Science. 2009; 324(5930):1029-33). This alternative energy pathway can be very favorable to the body in recovering from pathological events, but at the conclusion of the event (such as at the end of infection), if it does not turn off, it can affect fatigue levels. Chronic Fatigue Syndrome patients have been shown to have activated this alternative energy pathway, increasing the amount of energy that is produced by glycolysis in the cytosol that continues after their pathological incident has passed. (Lawson N, Hsieh C H, March D, Wang X. Elevated Energy Production in Chronic Fatigue Syndrome Patients. J Nat Sci. 2016; 2(10); Morris G, Maes M. Oxidative and Nitrosative Stress and Immune-Inflammatory Pathways in Patients with Myalgic Encephalomyelitis (ME)/Chronic Fatigue Syndrome (CFS). Curr Neuropharmacol. 2014; 12(2):168-85).

Nicotinamide adenine dinucleotide (NAD+) is a cofactor central to metabolism. NAD exists in two forms: an oxidized and reduced form, abbreviated as NAD+ and NADH respectively. NAD is involved in redox reactions, carrying electrons from one reaction to another. The NAD+/NADH ratio in the cell is a measure of the redox state of the cell.

Nuclear factor kappa-light-chain-enhancer of activated B cells (NF-κB) is a protein complex that under non-stressed conditions mostly locates in the cytoplasm of the cell. Upon activation via stress events (such as infection), NF-κB trans-locates to the nucleus where it controls transcription of DNA, resulting in cytokine production. NF-κB plays a key role in regulating the immune response to infection.

AMP-protein activated Kinase (AMPK) is a sensor and regulator of cellular energy homeostasis, a master switch regulating glucose and lipid metabolism and activation of AMPK, and results in many beneficial effects. (Misra, et al, The role of AMP kinase in diabetes, Indian J Med Res 125:389-398 (2007).

Thus, in an aspect of the disclosure, there is provided an oxaloacetate agent for the treatment of pathological fatigue, wherein said oxaloacetate is selected from the group consisting of
The oxaloacetate anion (in a salt) and/or
Oxaloacetic acid and/or
Enol-oxaloacetate and/or
Keto-oxaloacetate and/or
Hydrated oxaloacetate and/or
Anhydrous enol-oxaloacetate,
Or other synonym for oxaloacetate as recounted within this specification

According to one embodiment, said oxaloacetate agent for use in the treatment as described herein is in the pure oxaloacetate form.

According to one embodiment, said oxaloacetate agent for use in the treatment as described herein is in the form of a pharmaceutically acceptable salt thereof.

The use and production of oxaloacetate agents has been described in U.S. Pat. Nos. 10,137,099; 10,016,385; 9,561,199; 9,050,306 granted to the Inventor.

As stated above, the oxaloacetate agent may be a pharmaceutically acceptable salt. The term “pharmaceutically acceptable salt(s)”, as used herein, means those salts of compounds of the disclosure that are safe and effective for oral, subcutaneously, intramuscularly, or intravenously administration in mammals and that possess the desired biological activity. Pharmaceutically acceptable salts include salts of basic groups present in compounds of the invention.

As mentioned above, the present disclosure is based on the unexpected finding that the clinical outcome of treatment of disorders characterized by pathological fatigue is significantly improved by supplementation with oxaloacetate compounds.

In one embodiment, said administration of said oxaloacetate is at a dose of about 100 to about 6,000 mg per day, with the dose being dependent upon both the severity of the fatigue, and the time that the body has been in a pathologically fatigued state. In some embodiments, the dose of oxaloacetate is any of about 100 mg, 200 mg, 300 mg, 400 mg, 500 mg, 600 mg, 700 mg, 800 mg, 900 mg, 1000 mg, 2000 mg, 3000 mg, 4000 mg, 5000 mg or 6000 mg per day. In some embodiments, the dose of oxaloacetate is between any of about 100 mg and 200 mg, 200 mg and 300 mg, 300 mg and 400 mg, 400 mg, and 500 mg, 500 mg and 600 mg, 600 mg and 700 mg, 700 mg and 800 mg, 800 mg and 900 mg, 900 mg and 1000 mg, 1000 mg and 2000 mg, 2000 mg and 3000 mg, 3000 mg and 4000 mg, 4000 mg and 5000 mg or 5000 mg and 6000 mg per day.

In one embodiment, said administration of said oxaloacetate is ramped at a lower starting dose of about 100 to about 400 mg per day, and increased to about 1,000 to about 6,000 mg per day. In one embodiment, said administration of said oxaloacetate is ramped at a lower starting dose of about 100 mg, about 200 mg, about 300 mg or about 400 mg per day, and increased to about 1,000, about 2000 mg, about 3000 mg, about 4000 mg, about 5000 mg or about 6,000 mg per day. Some patients with altered metabolism associated with fatigue can have sleep problems when first taking large doses of oxaloacetate, as the excess energy they receive from the oxaloacetate may have problems dissipating, causing restless sleep. These patients may be ramped with smaller doses, for example 200 mg/day, held until their sleep pattern stabilizes to a minimum number of hours, and then increased to a higher level, and held again until their sleep pattern stabilizes to a minimum number of hours. This increase in dosage procedure can be repeated to increase the dosage a number of times, and may be especially important in ME/CFS patients that have had fatigue for several years.

In one embodiment, said administration of said oxaloacetate is dosed at about 1,000 mg to about 3,000 mg per day, then later reduced to a “maintenance dose” of about 100 to about 300 mg per day. In one embodiment, said administration of said oxaloacetate is dosed at about 1,000 mg, about 2000 mg or about 3,000 mg per day, then later reduced to a “maintenance dose” of about 100 mg, about 200 mg or about 300 mg per day. Discontinuation of the highest dose of oxaloacetate, for example 1,000 mg/day, can be suggested when patient derived fatigue questionnaires such as the Chalmers Fatigue Questionnaire, the Fatigue Severity Scale, or the PROMISE Fatigue Short Form 7A show reductions in fatigue that correlate with normal levels of fatigue seen in normal control groups. Fatigue can then be continually improved upon with a lower dose maintenance level, for example 200 mg oxaloacetate per day. A maintenance dose can be especially important as to prevent the recurrence of the pathological fatigue which has been noted in some patients.

To obtain high patient compliance, that is the degree to which a patient correctly follows medical advice, it is generally considered that the treatment regimens may not be complex to for a patient to be able to easily follow them. For example, it may be preferable that the administration of a drug is once, twice or three times a day, such as twice or once a day. Thus, in one embodiment, there is provided an oxaloacetate for use as described herein, wherein said oxaloacetate is administered once, twice or three times a day, such as once or twice a day. To clarify, the oxaloacetate dose may for example be administered orally twice a day in a dose of 500 mg, resulting in a daily dose of 1,000 mg. It will be appreciated, that said oxaloacetate may be administered a different number of times a day.

As disclosed in the Example section of the present disclosure, an oxaloacetate agent may be useful in the treatment of a disorder characterized by debilitating fatigue, which disorder often includes symptoms such as persistent and/or recurrent debilitating fatigue, diffuse musculoskeletal pain, sleep disturbances and subjective cognitive impairment. Non-limiting examples of such disorders include myalgic encephalomyelitis (ME), also known as chronic fatigue syndrome (CFS), which refers to a group of debilitating medical conditions characterized by persistent and debilitating fatigue, diffuse musculoskeletal pain, sleep disturbances, neuropsychiatric symptoms and cognitive impairment that last for a minimum of at least six months in adults. ME/CFS often occurs together with other diseases such as fibromyalgia (FM), multiple chemical sensitivities, irritable bowel syndrome and temporomandibular joint disorder. Additionally, several other disorders are also characterized by disabling fatigue. A non-limiting list of such diseases includes FM, mental fatigue, post stroke fatigue, Huntington's disease, Parkinson's disease, Alzheimer's Disease, Amyotrophic Lateral Sclerosis (ALS) multiple sclerosis, Myasthenia Gravis, narcolepsy, cancer, post cancer fatigue, ADHD, depression, post-viral fatigue, viral fatigue, post-bacterial infection fatigue, bacterial infection fatigue and combinations thereof. Additionally, fatigue may be associated with cancer with or without cytostatic treatment. Physical trauma can also cause fatigue that can be ameliorated with an oxaloacetate agent. The skilled person will appreciate that the disorder characterized by disabling fatigue may be a fatigue disorder or a pain disorder.

Thus, in one embodiment, there is provided an oxaloacetate agent as described herein for use in the treatment of a disorder characterized by persistent and debilitating fatigue, wherein said disorder is selected from the group consisting of myalgic encephalomyelitis/chronic fatigue syndrome, fibromyalgia, mental fatigue, post stroke fatigue, Huntington's disease, Parkinson's disease, Alzheimer's Disease, Amyotrophic Lateral Sclerosis, multiple sclerosis, Myasthenia Gravis, narcolepsy, cancer, post cancer fatigue, ADHD, depression post-viral fatigue, viral fatigue, post-bacterial infection fatigue, bacterial infection fatigue and fatigue associated with cancer with or without cytostatic treatment, depression and combinations thereof.

In one embodiment, said fatigue disorder is characterized by at least one of the conditions selected from fibromyalgia, mental fatigue, myalgic encephalomyelitis/chronic fatigue syndrome and depression. In another embodiment, said disorder is a pain disorder characterized by at least one of the conditions selected from of fibromyalgia, mental fatigue myalgic encephalomyelitis/chronic fatigue syndrome- and depression. In one embodiment, said disorder is ME/CFS. In one embodiment, said disorder is mental fatigue. In one embodiment, said disorder is depression and in another embodiment, said disorder is fibromyalgia. In one embodiment, said disorder is a combination of two or more above mentioned disorders, such as a combination selected from the group of: a combination of myalgic encephalomyelitis/chronic fatigue syndrome and fibromyalgia; a combination of myalgic encephalomyelitis/chronic fatigue syndrome and mental fatigue; a combination of myalgic encephalomyelitis/chronic fatigue syndrome and depression; a combination of mental fatigue and depression; a combination of fibromyalgia and depression; and a combination of mental fatigue and fibromyalgia. In one embodiment, said combination is selected from: a combination of myalgic encephalomyelitis/chronic fatigue syndrome, mental fatigue and fibromyalgia; a combination of myalgic encephalomyelitis/chronic fatigue syndrome, mental fatigue and depression; a combination of myalgic encephalomyelitis/chronic fatigue syndrome, depression and fibromyalgia; a combination of depression, mental fatigue and fibromyalgia.

The skilled person will appreciate that the embodiments discussed above in relation to the first aspect of the present disclosure, are equally relevant and applicable to multiple aspects disclosed herein.

In another aspect of the present disclosure, there is provided a pharmaceutical compound agent to normalize metabolism after injury or disease, wherein said dysfunction is selected from the group consisting of increased glycolysis, chronic activation of NF-κB, decreased NAD+/NADH ratio, mitochondrial insufficiency, and reduced activation of AMPK.

In one embodiment, said pharmaceutical composition further comprises at least one pharmaceutically acceptable excipient or carrier. Non-limiting examples of excipients includes diluents, disintegrants, binders, lubricants, glidants and agents that modify release of the active agent, such as polymers. The skilled person is aware of suitable excipients and carriers.

In another embodiment, said pharmaceutical composition further comprises at least one additional active agent. In one embodiment, said additional agent is an anti-fatigue agent, such as a stimulant, for example a caffeine-based stimulant or a central nervous system stimulating agent, such as methylphenidate and various amphetamine derivatives.

In one embodiment, there is provided a pharmaceutical composition as described herein comprising an amount of oxaloacetate about 100 mg to about 6,000 mg, such as about 200 to about 3,000 mg, such as approximately about 500 mg to about 1,000 mg. In some embodiments, there is provided a pharmaceutical composition as described herein comprising an amount of oxaloacetate of about any of 100 mg, 200 mg, 300 mg, 400 mg, 500 mg, 600 mg, 700 mg, 800 mg, 900 mg, 1000 mg, 2000 mg, 3000 mg, 4000 mg, 5000 mg or 6000 mg. In some embodiments, the pharmaceutical composition comprises between any of about 100 mg and 200 mg, 200 mg and 300 mg, 300 mg and 400 mg, 400 mg, and 500 mg, 500 mg and 600 mg, 600 mg and 700 mg, 700 mg and 800 mg, 800 mg and 900 mg, 900 mg and 1000 mg, 1000 mg and 2000 mg, 2000 mg and 3000 mg, 3000 mg and 4000 mg, 4000 mg and 5000 mg or 5000 mg and 6000 mg.

In one embodiment, said pharmaceutical composition is formulated for oral, subcutaneous, intramuscular, buccal, sublingual, suppository, transdermal or intravenous administration. As discussed above, it will be appreciated that non-invasive administration may be generally preferable. In one embodiment, said pharmaceutical composition is formulated for oral administration.

In one embodiment, wherein said pharmaceutical is formulated for oral administration, the pharmaceutical composition comprises approximately 100 mg to 6,000 mg, such as 200 to 3,000 mg, such as approximately 500 mg to 1,000 mg of an oxaloacetate. In some embodiments, the pharmaceutical composition formulated for oral administration comprises about any of 100 mg, 200 mg, 300 mg, 400 mg, 500 mg, 600 mg, 700 mg, 800 mg, 900 mg, 1000 mg, 2000 mg, 3000 mg, 4000 mg, 5000 mg or 6000 mg of an oxaloacetate. In some embodiments, the pharmaceutical composition formulated for oral administration comprises between any of about 100 mg and 200 mg, 200 mg and 300 mg, 300 mg and 400 mg, 400 mg, and 500 mg, 500 mg and 600 mg, 600 mg and 700 mg, 700 mg and 800 mg, 800 mg and 900 mg, 900 mg and 1000 mg, 1000 mg and 2000 mg, 2000 mg and 3000 mg, 3000 mg and 4000 mg, 4000 mg and 5000 mg or 5000 mg and 6000 mg of an oxaloacetate.

When said pharmaceutical is formulated for subcutaneous or intramuscular administration, it may be suitable that the administered dose corresponds to approximately half of the oral dose. Thus, in one embodiment wherein said pharmaceutical is formulated for subcutaneously or intramuscularly administration, the pharmaceutical composition comprises approximately 50 mg to 3,000 mg, such as 100 to 1,500 mg, such as 250 mg to 500 mg of an oxaloacetate. In some embodiments, the pharmaceutical composition formulated for subcutaneous or intramuscular administration comprises about any of 50 mg, 100 mg, 150 mg, 200 mg, 250 mg, 300 mg, 350 mg, 400 mg, 450 mg, 500 mg, 600 mg, 700 mg, 800 mg, 900 mg, 1000 mg, 2000 mg or 3000 mg of an oxaloacetate. In some embodiments, the pharmaceutical composition formulated for subcutaneous or intramuscular administration comprises between any of about 50 mg and 100 mg, 100 mg and 150 mg, 150 mg and 200 mg, 200 mg and 250 mg, 250 mg and 300 mg, 350 mg and 400 mg, 400 mg and 450 mg, 450 mg and 500 mg, 500 mg and 600 mg, 600 mg and 700 mg, 700 mg and 800 mg, 800 mg and 900 mg, 900 mg and 1000 mg, 1000 mg and 2000 mg, or 2000 mg and 3000 mg of an oxaloacetate.

In another embodiment, wherein said pharmaceutical composition is formulated for intravenous administration, the pharmaceutical composition comprises approximately 100 to 500 mg of an oxaloacetate. In some embodiments, the pharmaceutical composition formulated for intravenous administration comprises about any of 100 mg, 150 mg, 200 mg, 250 mg, 300 mg, 350 mg, 400 mg, 450 mg, or 500 mg of an oxaloacetate. In some embodiments, the pharmaceutical composition formulated for oral administration comprises between any of about 100 mg and 150 mg, 150 mg and 200 mg, 200 mg and 250 mg, 250 mg and 300 mg, 300 mg and 400 mg, 400 mg and 450 mg, or 450 mg and 500 mg of an oxaloacetate.

In one embodiment, there is provided a pharmaceutical composition formulated as a pill, tablet, capsule, dragee, liquid, gel capsule, syrup, slurry or suspension, such as a pill.

In one embodiment, there is provided a pharmaceutical composition formulated for administration once, twice or three times a day, such as once or twice a day.

In an additional aspect of the present disclosure, there is provided the use of an oxaloacetate agent as defined herein for the manufacture of a medicament for the treatment a disorder characterized by persistent and debilitating fatigue, such as a disorder disclosed herein.

### Pharmaceutical Compositions

### Pharmaceutical Preparations and Methods of Administration

Oxaloacetate can be administered to an individual at therapeutically effective doses for the prevention or treatment of fatigue disorders including such as ME/CFS, FM, mental fatigue, post stroke fatigue, Huntington's disease, Parkinson's disease, Alzheimer's Disease, Amyotrophic Lateral Sclerosis (ALS) multiple sclerosis, Myasthenia Gravis, narcolepsy, cancer, post cancer fatigue, ADHD, depression, post-viral fatigue, viral fatigue, post-bacterial infection fatigue, bacterial infection fatigue and combinations thereof.

As used herein, “oxaloacetate or OAA” includes oxaloacetic acid, the salt of the acid, or oxaloacetate in a buffered solution, anhydrous enol-oxaloacetate, enol-oxaloacetate in solution, keto-oxaloacetate in solution, hydrated oxaloacetate in solution as well as mixtures thereof. It also includes synonyms of oxaloacetate as shown herein.

### Effective Dose

A therapeutically effective dose refers to that amount of oxaloacetate sufficient to result in the desired effect such as the amelioration of symptoms relating to fatigue.

Toxicity and therapeutic efficacy of oxaloacetate can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. The LD₅₀ of oxaloacetate is above 5 g/kg of body weight. The “no observable adverse effects level” (NOAEL) in a 90-day sub-chronic rat study was 500 mg/kg (the highest dose in the test). Oxaloacetate has an exceptionally low toxicity, as would be expected from a chemical involved in the Citric Acid Cycle of every cell.

Toxicity studies of oxaloacetate run in Japan in 1968 on rats indicates that levels of oxaloacetate at 83 mg/kg of body weight caused changes in pancreatic islets. Some islets were decreased in size and hyperemic, alpha cells being atrophic, while beta cells were hypertrophic and stained densely. At lower doses, 41 mg/kg of body weight, the pancreas of the rates only demonstrated proliferation and hyperplasia of the islet cells. The liver, hypophysis, adrenals and gonadal glands showed no particular changes (Yoshikawa, Anti-diabetic effect of sodium oxaloacetate, 1968 Tohoku Journal of Experimental Medicine).

Doses of up to 6,000 mg/day for one year of anhydrous enol-oxaloacetate in a patient have resulted in no negative side effects.

An example of an effective dose of oxaloacetate administered by an intravenous injection is from between about 0.5 mg to about 1 g of oxaloacetate for each kg of body weight. In a preferred embodiment, the effective dose of oxaloacetate is between about 2.0 mg and about 40 mg for each kg of body weight. In some embodiments, the effective dose of the oxaloacetate compound is between any of about 2 mg/kg and 5 mg/kg, 5 mg/kg and 10 mg/kg, 10 mg/kg and 15 mg/kg, 15 mg/kg and 20 mg/kg, 20 mg/kg and 25 mg/kg, 25 mg/kg and 30 mg/kg, 30 mg/kg and 35 mg/kg, or 35 mg/kg and 40 mg/kg body weight. Due to the acidity of the compound, the effective dose can be administered in multiple injections over several hours, or continuously. Effective oral dosing would likewise range from about 0.5 mg to about 1 g of oxaloacetate for each kg of body weight with the preferred effective dosage range between about 2 mg to about 40 mg of oxaloacetate for each kg of body weight. For example, an adult male weighing approximately 80 kg would be administered between about 150 mg to about 3.5 g of oxaloacetate orally per day. Dermally, topical formulations comprising concentrations of about 0.5 to 16 mM of oxaloacetate are effective. Calorie Restriction (CR) studies indicate that restricting calories every-other-day yields the same beneficial results as daily CR. Similarly, in some embodiments, oxaloacetate is administered every-other-day, as once metabolism is re-normalized, the anti-fatigue effect lasts for at least a two-day period. In other embodiments, oxaloacetate is administered 3 times per day after each meal.

### Formulations

Pharmaceutical compositions for use in accordance with the present invention may be formulated in conventional manner using one or more physiologically acceptable carriers or excipients. Thus, oxaloacetate and its physiologically acceptable salts and solvates may be formulated for administration by inhalation or insufflation (either through the mouth or the nose) or oral, buccal, topical, transdermal, parenteral, or rectal administration.

Oxaloacetate is acidic. The acidity is unlikely to affect organisms that ingest the compound in beneficial amounts as the interior conditions of the stomach are also very acidic. The acidity may affect other tissues, including but not limited to the skin or lungs, that may benefit from the direct application of oxaloacetate. Therefore, in another embodiment, a composition of matter can be created by mixing oxaloacetate with a buffer solution or a base or used as a salt of oxaloacetate, so the delivered compound is not caustic. This will enable higher concentrations of oxaloacetate to be delivered safely to the organism, especially if the oxaloacetate is not delivered by oral ingestion.

For oral administration, the pharmaceutical compositions may take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycollate); or wetting agents (e.g., sodium lauryl sulphate). The tablets may be coated by methods well known in the art. Liquid preparations for oral administration may take the form of, for example, non-water solutions, syrups or suspensions, or they may be presented as a dry product for constitution with water or other suitable vehicle immediately before use (due to decarboxylation concerns). Water acts as a catalyst which allows for the conversion of solid enol-oxaloacetate to convert to the liquid keto-oxaloacetate form which spontaneously decarboxylates into pyruvate and carbon dioxide. Such non-water liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

While the absorption of oxaloacetate from the digestive tract will increase the entire organism's oxaloacetate levels, the immediate contact of oxaloacetate to the cells in the digestive tract will preferentially be in contact with the digestive tract cells, allowing the reduction in gastric diseases such as colon cancer, even if the ingested amounts of oxaloacetate are insufficient to provide benefit to the entire organism.

Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The topical pharmaceutical and cosmetic compositions of the present invention may be made into a wide variety of product types. These include, but are not limited to lotions, creams, beach oils, gels, sticks, sprays, ointments, pastes, mousses and cosmetics. These product types may comprise several types of pharmaceutical or cosmetic carrier systems including, but not limited to solutions, emulsions, gels and solids. The topical pharmaceutical and cosmetic compositions of the present invention formulated as solutions typically include a pharmaceutically acceptable organic solvent. The terms “pharmaceutically-acceptable organic solvent” refers to a solvent which is capable of having dissolved therein the oxaloacetate, and possesses acceptable safety properties (e.g., irritation and sensitization characteristics). Examples of a suitable pharmaceutically acceptable organic solvent include, for example, monohydric alcohols, such as ethanol, and polyhydric alcohols, such as glycols. If the topical pharmaceutical and cosmetic compositions of the present disclosure are formulated as an aerosol and applied to the skin as a spray-on, a propellant is added to a solution composition.

A type of product that may be formulated from a solution carrier system is a cream or ointment. An ointment can comprise a simple base of animal or vegetable oils or semi-solid hydrocarbons (oleaginous). An ointment can include from about 0.10% to about 2% of a thickening agent. Examples of suitable thickening agents include: cellulose derivatives (e.g., methyl cellulose and hydroxy propylmethylcellulose), synthetic high molecular weight polymers (e.g., carboxyvinyl polymer and polyvinyl alcohol), plant hydrocolloids (e.g., karaya gum and tragacanth gum), clay thickeners (e.g., colloidal magnesium aluminum silicate and bentonite), and carboxyvinyl polymers (CARBOPOLS®; sold by B. F. Goodrich Company, such polymers are described in detail in Brown, U.S. Pat. No. 2,798,053, issued Jul. 2, 1975). A more complete disclosure of thickening agents useful herein can be found in Sagarin, Cosmetics, Science and Technology, 2nd Edition, Vol. 1, pp. 72-73 (1972). If the carrier is formulated as an emulsion, from about 1% to about 10%, for instance, from about 2% to about 5%, of the carrier system comprises an emulsifier. Suitable emulsifiers include nonionic, anionic or cationic emulsifiers. Exemplary emulsifiers are disclosed in, for example, McCutcheon's Detergents and Emulsifiers, North American Edition, pages 317-324 (1986). Preferred emulsifiers are anionic or nonionic, although other types can also be employed.

An emulsion carrier system useful in the topical pharmaceutical and cosmetic compositions of the present disclosure is a microemulsion carrier system. Such a system preferably comprises from about 9% to about 15% squalane; from about 25% to about 40% silicone oil; from about 8% to about 20% of a fatty alcohol; from about 15% to about 30% of polyoxyethylene sorbitan mono-fatty acid (commercially available under the trade name Tweens) or other nonionics; and from about 7% to about 20% water. This carrier system is combined with the therapeutic agents described above, with the oxaloacetate carried in the non-water portion.

The topical pharmaceutical and cosmetic compositions of the present disclosure can also include a safe and effective amount of a penetration enhancing agent. Other conventional skin care product additives may also be included in the compositions of the present invention. For example, collagen, elastin, hydrolysates, primrose oil, jojoba oil, epidermal growth factor, soybean saponins, mucopolysaccharides, and mixtures thereof may be used. Various vitamins can also be included in the compositions of the present invention. For example, Vitamin A, and derivatives thereof, Vitamin B2, biotin, pantothenic, Vitamin D, and mixtures thereof can be used.

In yet a further embodiment of the current invention, the oxaloacetate delivered topically can be mixed with a penetration enhancing agent such as dimethylsulfoxide (DMSO), combinations of sucrose fatty acid esters with a sulfoxide or phosphoric oxide, or eugenol, that allows faster migration of the oxaloacetate into the dermal tissues and then further into deeper cellular tissues.

In one embodiment, the disclosed compounds are administered through a topical delivery system. Implantable or injectable polymer matrices, and transdermal formulations, from which active ingredients are slowly released are also well known and can be used in the disclosed methods. The controlled release components described above can be used as the means to deliver the disclosed compounds. The compositions can further include components adapted to improve the stability or effectiveness of the applied formulation, such as preservatives, antioxidants, skin penetration enhancers and sustained release materials. Examples of such components are described in the following reference works hereby incorporated by reference: Martindale—The Extra Pharmacopoeia (Pharmaceutical Press, London 1993) and Martin (ed.), Remington's Pharmaceutical Sciences.

Controlled release preparations can be achieved using polymers to complex or absorb oxaloacetate. The controlled delivery can be exercised by selecting appropriate macromolecule such as polyesters, polyamino acids, polyvinylpyrrolidone, ethylenevinyl acetate, methylcellulose, carboxymethylcellulose, and protamine sulfate, and the concentration of these macromolecule as well as the methods of incorporation are selected in order to control release of active compound.

In another embodiment, transdermal patches, steady state reservoirs sandwiched between an impervious backing and a membrane face, and transdermal formulations, can also be used to deliver oxaloacetate. Transdermal administration systems are well known in the art. Occlusive transdermal patches for the administration of an active agent to the skin or mucosa are described in U.S. Pat. Nos. 4,573,996, 4,597,961 and 4,839,174, which are hereby incorporated by reference. One type of transdermal patch is a polymer matrix in which the active agent is dissolved in a polymer matrix through which the active ingredient diffuses to the skin. Such transdermal patches are disclosed in U.S. Pat. Nos. 4,839,174, 4,908,213 and 4,943,435, which are hereby incorporated by reference. In one embodiment, the steady state reservoir carries doses of oxaloacetate in doses from about 2 mg to 40 mg per day.

Present transdermal patch systems are designed to deliver smaller doses over longer periods of time, up to days and weeks. A rate-controlling outer microporous membrane, or micro pockets of the disclosed oxaloacetate dispersed throughout a silicone polymer matrix, can be used to control the release rate. Such rate-controlling means are described in U.S. Pat. No. 5,676,969, which is hereby incorporated by reference. In another embodiment, the oxaloacetate is released from the patch into the skin of the patient in about 20-30 minutes or less.

These transdermal patches and formulations can be used with or without use of a penetration enhancer such as dimethyl sulfoxide (DMSO), combinations of sucrose fatty acid esters with a sulfoxide or phosphoric oxide, or eugenol. The use of electrolytic transdermal patches is also within the scope of the methods disclosed herein. Electrolytic transdermal patches are described in U.S. Pat. Nos. 5,474,527, 5,336,168, and 5,328,454, the entire contents of which are hereby incorporated by reference.

Oxaloacetate may be formulated for parenteral administration by injection, e.g., by bolus injection or continuous infusion. The injected oxaloacetate can be mixed with other beneficial agents prior to injection including but not limited to antibiotics and other medications, saline solutions, blood plasma, and other fluids. Immediate contact of elevated levels of oxaloacetate with the vascular system cells will result in the reduction in age-related diseases such as hardening of the arteries, even if the amounts of oxaloacetate are insufficient to provide age-related benefits to the entire organism. Formulations for injection may be presented in unit dosage form, e.g., in ampoules or in multi-dose containers, with an added preservative. The compositions may take such forms as suspensions, solutions or emulsions in oily or aqueous vehicles, and may contain formulator agents such as suspending, stabilizing and/or dispersing agents. Alternatively, the active ingredient may be in powder form for constitution with a suitable vehicle, e.g., sterile pyrogen-free water, before immediate use.

Oxaloacetate may also be formulated in rectal compositions such as suppositories or retention enemas, e.g., containing conventional suppository bases such as cocoa butter or other glycerides.

In addition to the formulations described previously, oxaloacetate may also be formulated as a depot preparation. Such long-acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

In yet still another embodiment, oxaloacetate can be mixed with animal foods for the treatment of fatigue in animals. Oxaloacetate can either be formulated as part of the animal food or administered separately as a supplement to the animal's food. As those skilled in the art know, dry pet foods, typically dry dog foods, normally contain protein, fat, fiber, non-fiber carbohydrates, minerals, vitamins and moisture components. For example, as major ingredients, there are typically one or two cereal grains, generally corn, wheat and/or rice. In addition, for a protein source they may contain poultry meal, by-product meat, meat and bone meal, or other animal or fish meal by-products. At times as well, grain protein supplements such as corn gluten, soybean meal or other oil seed meals can be added. In addition to an effective amount of oxaloacetate of between about 0.01% to 0.1% by weight of the chow, animal chow of the present invention additionally includes the following: typical nutrient content in the food dry matter includes crude protein from 14% to 50%, usually 20% to 25%; crude fat from 5% to 25%; and crude fiber usually is present in the range of from about 3% to 14%, usually about 5% to 7%, with the total mineral or ash content being within the range of 3% to 10%, usually 4% to 7%. The important point is not the precise formulation of the pet food, since many conventional and satisfactory ones for use in conjunction with the present invention are available on the market. Rather, the key to success is that enough oxaloacetate component be added to pet food rations, whichever formulation is used, to provide the oxaloacetate activity level at the ranges necessary for AMPK activation to support the prevention or treatment of disorders with pathological fatigue in animals.

### Measurements of Clinical Outcome

As described in the Example section to follow, the clinical outcome of administration of an oxaloacetate agent to a subject suffering from a disorder as described herein to a subject may be evaluated by the following tests and questionnaires. The skilled person is aware of the applicability of these test for the evaluation of fatigue and depression related symptoms.

As used herein, the term “Clinical Global Impression” (CGI) refers to a rating scale commonly used to measure symptom severity, treatment response and the efficacy of treatments in treatment studies of patients with mental disorders (Guy W: Clinical Global Impressions (CGI) Scale. Modified From: Rush J, et al.: Psychiatric Measures, APA, Washington D.C., 2000).

As used herein “Clinical Global Impression of Change” (CGI-C) (also known as Clinical Global Impression-Improvement (CGI-I)) scale is a 7-point scale that requires the clinician to assess how much the patient's illness has improved or worsened relative to a baseline state at the beginning of the intervention. The ratings are as follows: 1, very much improved; 2, much improved; 3, minimally improved; 4, no change; 5, minimally worse; 6, much worse; or 7, very much worse.

As used herein, the term “MFS” refers to a the mental fatigue self-assessment questionnaire (Johansson B et al (2010) Brain Injury 2010; 24:2-12).

Additionally, the clinical outcome of the treatment may be evaluated using the FF-scale, The Beck/BDI scale, VAS pain scale and by neuropsychological tests.

As used herein, the term “FF-scale” or “FF” refers to the FibroFatigue scale also known as the fibromyalgia and chronic fatigue syndrome rating scale described in by Zachrisson and coworkers (Zachrisson O, et al, (2002) J Psychosom Res Jun; 52(6):501-9). The FibroFatigue scale is an observer's rating scale with 12 items measuring pain, muscular tension, fatigue, concentration difficulties, failing memory, irritability, sadness, sleep disturbances, and autonomic disturbances and irritable bowel, headache and subjective experience of infection.

As used herein, the terms “Beck/BDI scale” and “BD” refers to the Beck Depression Inventory created by Aaron T. Beck (Beck A T et al., (1961) Arch. Gen. Psychiatry 4(6): 561-71). It is a 21-question multiple-choice self-report inventory and one of the most widely used instruments for measuring the severity of depression. The BDI questionnaire is designed for individuals aged 13 and over and is composed of items relating to symptoms of depression such as hopelessness and irritability, cognitions such as guilt or feelings of being punished, as well as physical symptoms such as fatigue, weight loss, and lack of interest in sex.

As used herein, the term “VAS pain scale” refers to the visual analog scale for measuring a patient's pain intensity or other features. The VAS scale is a psychometric response scale and is often used in questionnaires. It is a measurement instrument for subjective characteristics or attitudes that cannot be directly measured. When responding to a VAS item, respondents specify their level of agreement to a statement by indicating a position along a continuous line between two endpoints.

As used herein, the term “neuropsychological tests” refers to tests designed to measure unobserved constructs, also known as latent variables. Psychological tests are typically, but not necessarily, a series of tasks or problems that the respondent must solve and measure a respondent's maximum performance.

As used herein, the term “Chalder Fatigue Scale” refers to a fatigue questionnaire (Cella, M and T. Chalder (2010). “Measuring fatigue in clinical and community settings” J Psychosom Res 69(1):17-22) and is a validated patient self-assessment of fatigue.

As used herein, the term “Fatigue Severity Scale” refers to a fatigue questionnaire (Kleinman, L., Zodet, M. W., Hakim, Z., Aledort, J., Barker, C., Chan, K., Krupp, L., & Revicki, D. (2000). Psychometric evaluation of the fatigue severity scale for use in chronic hepatitis C. Quality of Life Research, 9, 499-508) and is a validated patient self-assessment of fatigue.

As used herein, the term “PROMIS Fatigue Short Form 7a” refers to a fatigue questionnaire (Christodoulou C, Schneider S, Junghaenel D U, Broderick J E, Stone A A. Measuring daily fatigue using a brief scale adapted from the Patient-Reported Outcomes Measurement Information System (PROMIS) Quality of Life Research. 2014; 23:1245-1253. doi: 10.1007/s11136-013-0553-z) and is a validated patient self-assessment of fatigue.

As used herein, the term “PDQ-39” refers to the “Parkinson's Disease Quality of Life 39 question survey. (Fitzpatrick R, Jenkinson C, Peto V, Hyman N, Greenhall R. Desirable properties for instruments assessing quality of life: evidence from the PDQ-39. J Neurol Neurosurg Psychiatry. 1997; 62(1):104).

As used herein, the term “MDS-UPDRS” refers to the “Movement Disorder Society-Sponsored Revision of the United Parkinson's Disease Rating Scale” (Goetz et al, Movement Disorder Society-Sponsored Revision of the United Parkinson's Disease Rating Scale (MDS-UPDRS): Scale Presentation and Clinimetric Testing Results, Movement Disorders Vol. 23, No. 15, 2008, pp. 2129-2170).

### Statistical Analysis

Various studies employed statistical evaluations of obtained data. The skilled person is aware of and knows how to employ the tests used herein. Any deviations from standard calculations procedures are explained in the Example section of the disclosure. Briefly, the statistics test employed herein are as follows:

The Mann-Whitney U test (also called the Mann-Whitney-Wilcoxon (MWW), Wilcoxon rank-sum test (WRS), or Wilcoxon-Mann-Whitney test) is a nonparametric test of the null hypothesis that two populations are the same against an alternative hypothesis, especially that a particular population tends to have larger values than the other.

The 2-way interaction analysis (2-way analysis of variance (ANOVA)) is a test that examines the influence of two different categorical independent variables on one continuous dependent variable. The two-way ANOVA not only aims at assessing the main effect of each independent variable but also at assessing if there is any interaction between them.

The 3-way interaction analysis (3-way analysis of variance (ANOVA)) is a test that examines if there is a 2-way interaction that varies across levels of a third variable.

Spearman's rank correlation coefficient is a nonparametric measure of statistical dependence between two variables. It assesses how well the relationship between two variables can be described using a monotonic function. If there are no repeated data values, a perfect Spearman correlation of +1 or −1 occurs when each of the variables is a perfect monotone function of the other.

Student's T-Test is a statistical hypothesis test in which the test statistic follows a Student's t-distribution under the null hypothesis. The t-test can be used, for example, to determine if the means of two sets of data are significantly different from each other.

While the invention has been described with reference to various exemplary aspects and embodiments, it will be understood by those skilled in the art that various changes may be made, and equivalents may be substituted for elements thereof without departing from the scope of the invention. Therefore, it is intended that the invention is not limited to any particular embodiment contemplated, but that the invention will include all embodiments falling within the scope of the appended claims.

### Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

As used herein, the singular form “a”, “an”, and “the” includes plural references unless indicated otherwise.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictates otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, “treatment” or “treating” is an approach for obtaining beneficial or desired results, including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. The methods of the invention contemplate any one or more of these aspects of treatment.

As used herein, the term “prophylactic treatment” refers to treatment, wherein an individual is known or suspected to have or be at risk for having a disorder but has displayed no symptoms or minimal symptoms of the disorder. An individual undergoing prophylactic treatment may be treated prior to onset of symptoms.

As used herein, by “combination therapy” is meant that a first agent be administered in conjunction with another agent. “In conjunction with” refers to administration of one treatment modality in addition to another treatment modality, such as administration of a composition of nucleated cells as described herein in addition to administration of an immunoconjugate as described herein to the same individual. As such, “in conjunction with” refers to administration of one treatment modality before, during, or after delivery of the other treatment modality to the individual.

The term “simultaneous administration,” as used herein, means that a first therapy and second therapy in a combination therapy are administered with a time separation of no more than about 15 minutes, such as no more than about any of 10, 5, or 1 minutes. When the first and second therapies are administered simultaneously, the first and second therapies may be contained in the same composition (e.g., a composition comprising both a first and second therapy) or in separate compositions (e.g., a first therapy in one composition and a second therapy is contained in another composition).

As used herein, the term “sequential administration” means that the first therapy and second therapy in a combination therapy are administered with a time separation of more than about 15 minutes, such as more than about any of 20, 30, 40, 50, 60, or more minutes. Either the first therapy or the second therapy may be administered first. The first and second therapies are contained in separate compositions, which may be contained in the same or different packages or kits.

As used herein, the term “concurrent administration” means that the administration of the first therapy and that of a second therapy in a combination therapy overlap with each other.

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug Administration.

### EXEMPLARY EMBODIMENTS

Embodiment 1. A method of treating one or more symptoms of a disorder characterized by debilitating fatigue in a subject, the method comprising administering a therapeutic amount of an oxaloacetate compound to the subject; wherein said disorder is selected from the group consisting of post-COVID-19 fatigue, post-viral fatigue, myalgic encephalomyelitis/chronic fatigue syndrome, fibromyalgia, mental fatigue, post-stroke fatigue, Amyotrophic Lateral Sclerosis, Myasthenia Gravis, Huntington's disease, Parkinson's disease, Alzheimer's Disease, multiple sclerosis, narcolepsy, post cancer fatigue, fatigue associated with cancer with or without cytostatic treatment.

Embodiment 2. The method of embodiment 1, wherein said oxaloacetate compound is an anhydrous enol-oxaloacetate.

Embodiment 3. The method of embodiment 1, wherein said oxaloacetate is comprised from the group of enol-oxaloacetate, keto-oxaloacetate, hydrated oxaloacetate, or an oxaloacetate salt.

Embodiment 4. The method of any one of embodiments 1-3, wherein said disorder is selected from the group consisting of fibromyalgia, mental fatigue, Myalgic encephalomyelitis/chronic fatigue syndrome, fibromyalgia, Huntington's disease, post-COVID-19 fatigue, post-viral fatigue, Amyotrophic Lateral Sclerosis, Myasthenia Gravis, Parkinson's disease, Alzheimer's Disease, multiple sclerosis, and post-cancer fatigue.

Embodiment 5. The method of any one of embodiments 1-4, wherein said oxaloacetate agent is administered in a dose of approximately 100 to 6,000 mg.

Embodiment 6. The method of any one of embodiments 1-5, wherein said oxaloacetate agent is administered in a dose of 200 mg to 3,000 mg.

Embodiment 7. The method of any one of embodiments 1-6, wherein said oxaloacetate agent is administered once, twice or three times a day.

Embodiment 8. The method of any one of embodiments 1-7, wherein the oxaloacetate compound is in a pharmaceutical composition.

Embodiment 9. A method of treating one or more symptoms of a disorder characterized by debilitating fatigue in a subject, the methods comprising administering a therapeutic amount of a compound to reverse metabolic dysfunction to the subject; wherein said dysfunction is selected from the group consisting of increased glycolysis, chronic activation of NF-kB, decreased NAD+/NADH ratio, mitochondrial insufficiency, reduced activation of AMPK, and combinations thereof.

Embodiment 10. The method of embodiment 9 wherein said compound is an oxaloacetate compound.

Embodiment 11. The method of embodiment 10, wherein said oxaloacetate compound is selected from the group consisting of enol-oxaloacetate, keto-oxaloacetate, hydrated oxaloacetate and an oxaloacetate salt.

Embodiment 12. The method of embodiment 10 or 11, wherein said oxaloacetate compound is anhydrous enol oxaloacetate

Embodiment 13. The method of any one of embodiments 9-12, wherein said disorder is selected from the group consisting of fibromyalgia, mental fatigue, Myalgic encephalomyelitis/chronic fatigue syndrome, fibromyalgia, Huntington's disease, post-COVID-19 fatigue, post-viral fatigue, Amyotrophic Lateral Sclerosis, Myasthenia Gravis, Parkinson's disease, Alzheimer's Disease, multiple sclerosis, and post-cancer fatigue.

Embodiment 14. The method of any one of embodiments 10-13, wherein said oxaloacetate compound is administered in a dose of about 100 to about 6,000 mg.

Embodiment 15. The method of any one of embodiments 10-14, wherein said oxaloacetate compound is administered in a dose of about 200 mg to about 3,000 mg.

Embodiment 16. The method of any one of embodiments 10-15, wherein said oxaloacetate compound is administered once, twice or three times a day.

Embodiment 17. The method of any one of embodiments 9-16, wherein the compound to reverse metabolic dysfunction is in a pharmaceutical composition.

### EXAMPLES

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### Example 1

Patients with Parkinson's disease have significant fatigue. It is their number one complaint. Here is text taken from the Parkinson.Org website on Jan. 5, 2020, a leading organization for patients with Parkinson's Disease that discuses Parkinson's pathological fatigue:
Do you or a loved one with Parkinson's disease (PD) feel physically or mentally exhausted? This could be fatigue? a feeling of deep tiredness that does not improve with rest. About half of people with PD report fatigue is a major problem and a third say it is their most disabling symptom.
Fatigue is different from sleepiness. A person who is fatigued feels exhausted, however, does not necessarily feel like sleeping.
Fatigue is common early in the course of PD, but can occur at any point and can happen whether movement symptoms are mild or severe. It is sometimes confused with other symptoms that can make a person sleepy or tired, like sleep disturbances or pain. Fatigue is also a symptom of depression, but a person can be fatigued without being depressed. Stress can make fatigue worse.
No specific cause has been shown to cause fatigue in PD. It is possible that motor symptoms like tremor and stiffness contribute to making muscles tired. But fatigue can have causes outside of Parkinson's, too. It is important to identify and treat illnesses or medications not related to PD that cause fatigue.
The extreme exhaustion that comes with fatigue can lead people to reduce hours at work or retire, or avoid social activities. Understanding fatigue as a symptom of PD and finding ways to cope with it are essential to maintaining a good quality of life.

Symptoms
Physical fatigue: feeling deeply tired or weary; may worsen with “off” fluctuations.
Mental fatigue: mental tiredness that makes it difficult to concentrate.

Therapies
Few therapies have been tested for fatigue in PD. None have been proven effective against fatigue on its own. Therapies for movement symptoms do not seem to help fatigue.

Taken from the world wide web at parkinson.org/Understanding-Parkinsons/Symptoms/Non-Movement-Symptoms/Fatigue on Jan. 5, 2020

The inventor set up an initial set of case studies to investigate the effects of oxaloacetate on Parkinson's disease. Several people in the case studies reported a decrease in fatigue with the use of an Oxaloacetate nutritional supplement, “benaGene” manufactured and distributed by the Inventor. Here is the testimonial of one such person:
Alan,
I will try to answer all of your questions as clearly as I can.
I take one benaGene at breakfast and one with dinner, so they are about 10-12 hours apart, and I take the Magnesium at about the same time as the benaGene. The Mg2+ is in the oxide form and each capsule has 400 mg. Apparently, the oxide is not the best absorbed form, but it is the only one we have been able to find in the pharmacy.
The reason I went to a neurologist initially was that I felt like I was having memory problems beyond those associated with normal aging (beginning in the early Fall of 2009). This presented great difficulties because I am a faculty member at KU and taught Cardiovascular Pharmacology for many years. I could tell that my speaking was becoming very halted and slow as I was searching for words. At about the same time, I began having vigorous muscular spasms when I was sleeping. This phenomenon is known as Rem Sleep Without Atonia (RSWA) or REM sleep abnormal behaviors, a condition that has been studied by scientists at the Mayo Clinic in Minnesota. As I recall, the next problem that appeared was a significant disruption in visual-spatial coordination, and this led Eli to convince me I needed to stop driving. My balance was also deteriorating, and I fell several times. So, in December of 2009, I went to the Neurology Clinic at the Medical Center, thinking I might have Alzheimer's. The neurologist is an expert in Alzheimer's Disease, and I thought that might be what my problem was. However, he felt it was more likely that I had a form of Parkinson's Disease and arranged for me to see the neurologist who specializes in movement disorders. Although he did not consider the symptoms very severe at that time, he did put me on Sinemet (4 tablets per day) the commonly used drug that contains levo-dopa and carbidopa (100 mg L-DOPA, 25 mg Carbidopa). And that treatment certainly seemed to help me maintain better balance for a while. But I began to develop tremors in my hands and was no longer able to type on the computer keyboard, to carry anything without spilling it, to button my clothes, or to comb my hair. My handwriting was illegible, and I had great degree of difficulty in holding to silverware and feeding myself Although the neurologist kept increasing the amount of Sinemet I was taking (up to 7 tablets/day), the symptoms just got worse with time. In late March and early April, I had several bad falls, one of them leading to breaking of a bone in my hand. By April 14, I was up to 8 Sinemet tabs/day and still had not achieved any improvement. A visit to the neurologist on April 14 convinced him that I would definitely need a walker as I could only stand for 2.3 seconds without falling back on my chair.
Eli had been combing the literature on PD and was alerted to the idea that mitochondrial dysfunction is a major contributor to the pathogenesis of PD. And he also knew that Magnesium has very beneficial effects in animal models of various CNS problems and that it may counteract abnormal calcium surges in neurons. A neurologist friend who studies mitochondria alerted him to oxaloacetate and the existence of Terra Biologicals. So on May 9, 2011, I began taking the benaGene and Magnesium along with the Sinemet (7.5 tabs/day). After four weeks on benaGene/Mg combination, my balance and posture had improved sufficiently for us to consider, once again, going to scientific meetings in Europe. That would have been absolutely impossible in the state that I was in before this treatment regimen was initiated. Both my balance and flexibility improved. The tremors gradually began to subside and were almost completely gone by the time we returned from Europe (beginning of August). Both tremors and visual problems subsided to the point where I could give up my Voice-activated computer program. The improvement in my visual-spatial function enabled me to start driving my car by the end of August. I am now capable of typing on my computer, attending meetings of my research staff, and working on scientific manuscripts. I am still continuing on the same benaGene/Mg therapy and the Sinemet at 7 tabs/day.
I hope this gives you a good picture of the reversal of many of the PD symptoms by the benaGene/Mg treatment. Please feel free to contact me if you have any other questions.
Mary

As can be seen, this person saw an improvement in pathological muscle fatigue, reducing tremors that can occur due to muscle fatigue. One patient's testimonial is important, especially in a disease such as Parkinson's where there is no cure. But to better examine efficacy, the inventor then implemented a retrospective study on the effects of the nutritional supplement benaGene (a combination of 100 mg oxaloacetate and 150 mg Vitamin C) on the Inventor's customer base-which included some Parkinson's patients. Each participant with Parkinson's Disease was given a “Parkinson's Disease Quality of Life (PDQ)” survey with 39 questions (PDQ-39) and was asked to score their historical Parkinson's Disease condition prior to taking benaGene (oxaloacetate) and then again after taking benaGene for at least 3 months. Before/After Data was received from 13 customers, and scores were compiled.

53.8% of the Parkinson's Disease persons taking benaGene (oxaloacetate) responded favorably with an improvement in quality of life as measured by the before/after survey with the PDQ-39. Of those that did respond favorably, the improvement in quality of life was greater than 50%, with some showing up to a 90% discontinuation of symptoms, including pathological fatigue. Quality of Life improvement includes improvement in fatigue.

### Example 2

Based on the findings of the Inventor, a clinical trial was performed on Parkinson's Disease patients. The trial used the MDS-UPDRS (Goetz et al, Movement Disorder Society-Sponsored Revision of the United Parkinson's Disease Rating Scale (MDS-UPDRS): Scale Presentation and Clinimetric Testing Results, Movement Disorders Vol. 23, No. 15, 2008, pp. 2129-2170) (45) as a validated method to clinically measure pathological fatigue in Parkinson's patients. The Fatigue question is as follows:
1.13 Fatigue
Over the past week, have you usually felt fatigued? This feeling is not part of being sleepy or sad
0: Normal: No fatigue
1: Slight: Fatigue occurs. However, it does not cause me troubles doing things or being with people.
2: Mild: Fatigue causes me some troubles doing things or being with people.
3: Moderate: Fatigue causes me a lot of troubles doing things or being with people.
However, it does not stop me from doing anything.
4: Severe: Fatigue stops me from doing things or being with people.

26 patients completed the Phase 2 study with 13 patients in the Placebo group, and 13 patients in the oxaloacetate group.

Our initial data to support the clinical trial on Parkinson's Patient's came from our “benaGene” studies discussed in Example 1 of this specification. benaGene is a nutritional supplement of 100 mg oxaloacetate and 150 mg ascorbic acid (Vitamin C) in a vegetable capsule shell. Because we did not know if the benefits seen in Example 1 above were due to ascorbic acid or due to oxaloacetate, ascorbic acid (Vitamin C) was selected as the placebo at 250 mg, whereas the active substance to be tested was 100 mg oxaloacetate/150 mg ascorbic acid capsules (benaGene).

The double blinded placebo-controlled trial measured the effect of 2 capsules of benaGene (oxaloacetate) per day for 4 months in the Parkinson's patients.

After 4 months, the oxaloacetate group showed significant statistical improvement in the fatigue question of the MDS-UPDRS scale over the initial baseline measurement. The oxaloacetate score for fatigue was improved by 28.7%, P value<0.05. In contrast, the ascorbic acid placebo group showed only a 10.4% improvement in fatigue, and P value was not significant at P=0.71.

200 mg Oxaloacetate was clinically proven to significantly reduce Pathological Fatigue in a double blinded, placebo-controlled clinical trial of Parkinson's patients with oxaloacetate supplied by the Inventor. Thus, oxaloacetate supplementation is a novel method to ameliorate pathological fatigue in Parkinson's Disease.

### Example 3

In one study of 384 patients that were discharged from the hospital after COVID-19 infection, 69% had continuing long-term Pathological fatigue. (Mandal S, Barnett J, Brill S E, Brown J S, Denneny E K, Hare S S, et al. ‘Long-COVID’: a cross-sectional study of persisting symptoms, biomarker and imaging abnormalities following hospitalization for COVID-19. Thorax. 2020). A physician skilled in the art of pathological fatigue recounts her experience with a post-COVID-19 significant persistent pathological fatigue. The physician contracted COVID-19 and after resolution of the viral infection, experienced extreme fatigue. Her fatigue was measured using validated fatigue measurement surveys, including the Chalder Fatigue Score, the Fatigue Severity Score, the Visual Fatigue Score, and the PROMIS Fatigue Short Form 7a. All measurements showed severe fatigue. The physician was placed on a course of 500 mg anhydrous enol-oxaloacetate BID for 45 days. Her fatigue scores were taken after two weeks and showed a slight improvement. After a total of six weeks, her fatigue was resolved. Her scores appear below in Table 1.

TABLE 1

| | Baseline | | Final | Fatigue Test Score Range | |
|---|---|---|---|---|---|
| | Oct. 14, | Nov. 3, | Dec. 3, | Fatigue | No |
| | 2020 | 2020 | 2020 | Worst | Fatigue |
| Chalder Fatigue | 22 | 16 | 2 | 33 | 0 |
| Score | | | | | |
| Fatigue Severity | 54 | 54 | 20 | 63 | 9 |
| Score | | | | | |
| Visual Fatigue | 3 | 4 | 10 | 0 | 10 |
| Score | | | | | |
| PROMIS | N/A | 25 | 11 | 35 | 7 |
| Fatigue 7a | | | | | |

The physician provided us with a testimonial concerning her experience:
I had been suffering from extreme fatigue for a few weeks and then I was offered a trial of oxaloacetate. I was initially skeptical because of the nausea that it caused but I decided to push through, and I must say I am glad that I did. By the end of six weeks, my energy is through the roof, I have never felt this happy or motivated or energized and clear minded before. My life has improved so much for the better. Thank you! LI

Because post-COVID-19 extreme fatigue may take up to several months to resolve (if at all), the physician's full reversal of severe fatigue in less than a 6-week period is an unexpected and novel event.

### Example 4

A physician customer of the Inventor used 5 capsules of benaGene (oxaloacetate) BID on a patient with pathological fatigue from a post-COVID-19 infection. The physician provided this report back to the Inventor:
I just received a positive report from a 50-year-old woman who survived COVID19 only to be left devastated with post-viral fatigue that had lasted 4 months. I had her try benaGene [AEO] and in 3 days she says the effects are dramatic. Yesterday she was feeling nearly like her pre-COVID self again. I've asked her to update me again in a month. I will let you know about how she's doing after taking benaGene/hydroxy fumarate for a little longer.

The Inventor checked in with the physician after 30 days to check on the patient. The physician reported:
She is still going strong . . . . She is the wife of another MD.

It is unexpected and novel that post-COVID-19 fatigue that has continued for 4 months can be improved within 3 days with oxaloacetate supplementation.

### Example 5

The 29-year old daughter of the Inventor contracted COVID-19 in late February 2020 and had a fever for 26 days. Afterwards, although her fever was resolved, she experienced fatigue so extreme that it was difficult for her to walk to the bathroom. She started taking 500 mg anhydrous enol-oxaloacetate BID for 45 days. Within 3 days 80% of fatigue was resolved. After 30 days, 100% of her fatigue was resolved.

It is unexpected and novel that post-COVID-19 extreme fatigue was reduced 80% in 3 days, and 100% within 30 days.

### Example 6

Patients with Amyotrophic Lateral Sclerosis (ALS) commonly experience pathological fatigue. (Gibbons C, Pagnini F, Friede T, Young C A. Treatment of fatigue in amyotrophic lateral sclerosis/motor neuron disease. The Cochrane database of systematic reviews. 2018; 1.CD011005).

A Phase 1 clinical trial was conducted with the Inventor supplying the anhydrous enol-oxaloacetate drug. Typically, Phase 1 clinical trials focus on the safety of a proposed drug, but patient response efficacy is also important and is noted. We present two testimonial from a patients who were taking 1500 mg anhydrous enol-oxaloacetate BID, who saw improvements in their pathological fatigue.
Patient 1. My name is Kevin Fairchild. I was diagnosed with Bulbar onset ALS by Dr. Jawdat at the KU Medical Center in Kansas City KS on Oct. 14, 2020. I was offered this trial of oxaloacetate by Dr. Jawdat taking 1500 mg twice daily for 28 days. Coming into the trial, my symptoms included slurred speech, my tongue didn't move well, and my left hand and arm are significantly atrophied. I have muscle fasciculations pretty much all over my body at times and most are constant. The first 4 to 5 days taking the supplement, I did feel like they intensified an hour to two hours after taking it, and it lasted for approximately an hour or two. After those 4 or 5 days, it went back to the normal strength. At approximately the third week of taking the supplement I began to notice my tongue movement had better range of motion. I could see it pushing out the side of my cheek, I couldn't get it over there before. I also have felt like my left hand has gained a little strength and grip and I feel like I have a bit more strength in my left wrist. I do however feel like most things are still declining, but with such a short time of trying the supplement, I want to keep taking it to see if things can keep improving/changing. I am currently not on any ALS medications but do take some vitamins.
Patient 2. My husband, Nathan, was part of a study with oxaloacetate for ALS. When he started the supplements [1500 mg Oxaloacetate BID], we did not expect a huge difference. However, within a couple of days, he was feeling much more energetic. Before taking oxaloacetate, he was struggling to get out of bed and was tired all the time. He would work during the day, but then be asleep in his recliner most of the evening. After a week of taking the medication, my 18 year old son commented that Dad was up and talking to him in the evenings. He said whatever he is doing, it is making a big difference. That really made an impact on me. Since the trial has finished, we are back to Nathan struggling to stay up and motivated to move. My kids and I have talked almost daily that we feel like the oxaloacetate made a positive difference in my husband's quality of life.

There is no approved medication to improve muscle fatigue in ALS patients, thus this result with oxaloacetate supplementation is unexpected and novel.

### Example 7

Patients with Amyotrophic Lateral Sclerosis (ALS) commonly experience pathological fatigue. (Gibbons C, Pagnini F, Friede T, Young C A. Treatment of fatigue in amyotrophic lateral sclerosis/motor neuron disease. The Cochrane database of systematic reviews. 2018; 1:CD011005).

A Phase 1 clinical trial was conducted with the Inventor supplying the anhydrous enol-oxaloacetate drug. Typically, Phase 1 trials focus on the safety of a proposed drug, but patient response is also important. We present a testimonial from a patient who was taking 1000 mg anhydrous enol-oxaloacetate BID, who saw improvement in his muscle fatigue.
Hi, my name is Matthew Grzesik, I recently participated in the TOALS study at the KU ALS clinic. Specifically, I was in the first group at the 2000 mg dose. Typically, I take between 2 and 4 long naps a day to combat relentless fatigue from ALS and have also had to moderate how much physical activity I do as to not wear myself out. Since I'm only 28 and was a strength and conditioning coach as well as a competitive powerlifter, limiting physical activity has been a struggle for me. I had planned to do a lawn renovation while I was participating in the study and was going to take my time, so I didn't overdo it. About a week into the study, I noticed that my energy levels were much higher than they've been in more than a year and I was able to get, what was going to be a month-long project, done in a week and a half Not only was I able to do more physically, but I also started to only need one nap a day, and eventually that went down to zero. It felt like a gift to feel as good as I did for that month. The only side effect I noticed was an upset stomach when I took my dose without food, however, it went away as soon as I ate something. I wanted to share something positive with you as well as ask for more Oxaloacetate so I can continue to take it and enjoy as much life as possible. Thank you for partnering with Dr. Jawdat for this study and for your time and consideration. I'm looking forward to seeing how this supplement can benefit others, whether that be with ALS patients, or other diseases.

There is no approved medication to improve muscle fatigue in ALS patients, thus this result with oxaloacetate supplementation is unexpected and novel.

An ALS patient who has been taking 3,000 mg of oxaloacetate daily, experienced no decline in function and had stable fatigue/muscle function after 8 months of supplementation. For ALS, this is surprising and welcome news as the anticipated decline rate for this period of time ranges from 12% to 43% as measured by the ALS FRS. (Ong M L, Tan P F, Holbrook J D. Predicting functional decline and survival in amyotrophic lateral sclerosis. PLoS One. 2017; 12(4):e0174925. Published 2017 Apr. 13. doi:10.1371/journal.pone.0174925).

The patient's email:
“Dear Alan, Good afternoon. I am writing to ask for more oxaloacetate for my husband, Darrell He has been taking the medication since May 2021 and has experienced no decline in his mobility or strength. We are thrilled! Could we please have some more medicine? Thanks!”

### Example 8

The Inventor has supplied Anhydrous Enol-Oxaloacetate for fatigue after pneumonia. Pneumonia patients often suffer from physiological fatigue. In a retrospective study of 506 adults with clinical and radiographic evidence of pneumonia, 510% of the patients suffered from fatigue at 90 days after diagnosis. (Metlay J P, Fine M J, Schulz R, Marrie T J, Coley C M, Kapoor W N, et al. Measuring symptomatic and functional recovery in patients with community-acquired pneumonia. J Gen Intern Med. 1997; 12(7):423-30). Below is the testimonial from a patient who found benefit from 500 mg oxaloacetate daily.
I resumed taking oxaloacetate shortly after having been released from the hospital and from skilled nursing care. The place where oxaloacetate made the most significant difference for me was in alleviation of the crushing fatigue that accompanied the pneumonia. The National Lung Association indicates that the basis of the fatigue is not understood, and certainly the scientific literature does not display any solid understanding of the biological causation of the fatigue.
The fatigue that I experienced can only be described as “crushing.” An example: shortly after being released to return home, I found that riding in a car (not driving) to drop someone at the airport and then stopping for lunch brought me to the point of complete exhaustion and required 2 full days for recovery. On querying nurses and physical therapists about this response, the universal answer was that it was “typical.” Speaking with people who had had pneumonia at different ages—granted that this is anecdotal—garnered consistent stories of extreme fatigue and a slow recovery that took years to achieve prior energy levels even with people who had been physically active teenagers at the time.
I was in the hospital for 2 weeks and then in skilled care for about 6 weeks, putting me in the 40% of the population that don't manage to avoid hospitalization. However, I hadn't been in the ICU, so it wasn't at the worst end of the spectrum.
When I took the first oxaloacetate capsule, I very quickly felt relief from a sensation I didn't even realize I was experiencing: a sense of an oppressively heavy weight in the center of my chest. That sensation left and never returned.
For the first few months, I chose to take multiple capsules during the course of the day. The oxaloacetate immediately raised my energy levels and enabled me to work from home. If I went into the office, I'd find that I could do well for several hours and, when fatigued, an oxaloacetate would prevent a rapid descent into exhaustion.
Of course, there is no way to judge whether oxaloacetate use sped up my recovery. But I can certainly tell you that it has played a noticeable role in alleviating the misery of the recovery process.

The rapid resolution of fatigue with oxaloacetate supplementation after Pneumonia is unexpected and novel.

### Example 9

A Phase 2 trial is currently underway to treat mental and physical fatigue in breast cancer survivors with an oxaloacetate agent and serves to teach a method to treat pathological mental and physical fatigue in cancer patients. In the test, fatigue will be assessed by a multidimensional fatigue symptom inventory. Scores will be summarized as means and standard deviations and confidence intervals will be calculated. Changes will also be summarized as effect sizes.

Inclusion Criteria for this clinical trial:
Women with a diagnosis of early-stage breast cancer (stage 0, I, II, IIIa) who are at least 12 months after surgery, chemotherapy, radiation, but may be on endocrine therapy or HER-2 targeted therapy, and not more than 5 years after their breast cancer diagnosis
No evidence of active/recurrent breast cancer or other serious chronic illness
Has significant cognitive complaints, defined as a score of <12 on the Patient-Reported Outcomes Measurement Information System (PROMIS) Adult version (v) 2.0-Cognitive Function 4a
Is geographically accessible, and able to participate in a study of 8-10 weeks duration
Ability to complete evaluation surveys in English
The effects of oxaloacetate on the developing human fetus at the recommended therapeutic dose are unknown. For this reason, women of childbearing potential must agree to use adequate contraception (barrier method of birth control; intrauterine device [IUD]; abstinence) prior to study entry and for the duration of study participation. Women of any age who have had their ovaries and/or uterus removed will not be at risk for pregnancy and will not require contraception. Should a woman become pregnant or suspect she is pregnant while participating in this study, she should inform her study physician immediately. Menopausal status will be established as follows: Women who are 55 years or older and who are not menstruating will be considered postmenopausal and not at risk for pregnancy. Women who are less than 55 years old who are menstruating will be considered premenopausal and will require contraception. Women who are less than 55 years with an intact uterus and ovaries who are not menstruating and have not had a menstrual period within the past 2 years will have a follicle-stimulating hormone (FSH) and estradiol measured. If the values are in postmenopausal range the woman will be considered postmenopausal and she will not be considered at risk for pregnancy.
Ability to understand and the willingness to sign a written informed consent document.

Exclusion Criteria:
Has another serious or chronic medical or psychiatric condition that contributes to substantial physical or emotional disability that would detract from participating in the planned study
Taking chronic medications that would interfere with cognitive functioning such as medications for sleep, anxiety, narcotics for pain, use of illicit drugs or cannabis
Participants may not be receiving any other investigational agents
History of allergic reactions attributed to compounds of similar chemical or biologic composition to oxaloacetate
Uncontrolled intercurrent illness including, but not limited to, ongoing or active infection, symptomatic congestive heart failure, unstable angina pectoris, cardiac arrhythmia, or psychiatric illness/social situations that would limit compliance with study requirements
Pregnant or breast-feeding women are excluded from this study because the safety of oxaloacetate in this setting is unknown. A pregnancy test will be performed on all women with an intact uterus and ovaries who are not determined to be postmenopausal.

Primary Outcome Measures:

1. Change in Functional Assessment of Cancer Therapy-Cognitive Function Perceived Cognitive Impairment (FACT-Cog PCI) score [Time Frame: Baseline up to day 57]

Patients who have an improvement in the FACT-Cog PCI of at least 4 points will be considered to have a positive response. The response proportion will be further characterized using a point estimate and 95% exact binomial confidence interval.

Secondary Outcome Measures:

1. Incidence of adverse events [Time Frame: Up to day 57]

Will be assessed by Common Terminology Criteria for Adverse Events version 4.

2. Fatigue [Time Frame: At day 57]

Will be assessed by Multidimensional Fatigue Symptom Inventory. Scores will be summarized as means and standard deviations and confidence intervals will be calculated. Changes will also be summarized as effect sizes to support design of a future trial.

3. Insomnia [Time Frame: At day 57]

Will be assessed by the Insomnia Severity Index. Scores will be summarized as means and standard deviations and confidence intervals will be calculated. Changes will also be summarized as effect sizes to support design of a future trial.

4. Depressive symptoms [Time Frame: At day 57]

Will be assessed by Patient-Reported Outcomes Measurement Information System. Scores will be summarized as means and standard deviations and confidence intervals will be calculated. Changes will also be summarized as effect sizes to support design of a future trial.

5. Neurocognitive tests [Time Frame: Up to day 57]

Subjects will be assessed by the California Verbal Learning Test-2nd edition, Brief Visuospatial Memory Test-Revised, Golden Stroop, Trail Making Test, Verbal Fluency, Connor's Continuous Performance Test-II. All scales listed will have the individual scores transformed by using published normative data, therefore the unit of measure will be the same for the listed tests; higher scores indicate better performance.

The clinical trial provides an example on how to use the oxaloacetate agent to treat cognitive and muscular fatigue in patients with breast cancer, which is a novel use of oxaloacetate.

### Example 10

A clinical trial evaluating the response of patients with post-COVID-19 fatigue treated by an oxaloacetate is currently underway. Fatigue is assessed by the Chalder Fatigue Scale, the Fatigue Severity Scale, and PROMIS-Fatigue-Short Form 7a. Scores are summarized as means and standard deviations and confidence intervals will be calculated. Changes are also summarized as effect sizes. The study is being performed as a double-blind placebo-controlled study, wherein half of the patients were administered the active drug and the other half were administered placebo. The trial is being conducted in agreement with the International Conference on Harmonization (ICH) guidelines on Good Clinical Practice (GCP). All patients provided written informed consent to participate in the study prior to being screened.

Inclusion Criteria:
4.1.1 Women with an initial infection diagnosis COVID-19 that has been resolved, as measured by rRT PCR, who are at least 2 months after proven viral resolution.
4.1.2 No evidence of active/recurrent COVID-19 or other serious chronic illness.
4.1.3 Have significant fatigue complaints, defined as a bimodal score of 4 or greater on the Fatigue Questionnaire.
4.1.4 Is geographically accessible, or can fill out forms virtually, and able to participate in a study of 6-10 weeks duration.
4.1.5 Age>18 years and less than 65.
4.1.6 Ability to complete evaluation surveys in English.
4.1.7 The effects of oxaloacetate on the developing human fetus at the recommended therapeutic dose are unknown. For this reason, women of childbearing potential must agree to use adequate contraception (barrier method of birth control; IUD; abstinence) prior to study entry and for the duration of study participation. Women of any age who have had their ovaries and/or uterus removed will not be at risk for pregnancy and will not require contraception. Should a woman become pregnant or suspect she is pregnant while participating in this study, she should inform her study physician immediately.
   Menopausal status will be established as follows: Women who are 55 years or older and who are not menstruating will be considered postmenopausal and not at risk for pregnancy. Women who are less than 55 years old who are menstruating will be considered premenopausal and will require contraception. Women who are less than 55 years with an intact uterus and ovaries who are not menstruating and have not had a menstrual period within the past 2 years will have an FSH and estradiol measured. If the values are in postmenopausal range the woman will be considered postmenopausal and she will not be considered at risk for pregnancy.
4.1.8 Ability to understand and the willingness to sign a written informed consent document.
4.1.9 No diagnosis of Clinical Depression
4.1.10 Is not taking an oxaloacetate supplement.

Exclusion Criteria:
4.2.1 Has another serious or chronic medical or psychiatric condition that contributes to substantial physical or emotional disability that would detract from participating in the planned study.
4.2.2 Taking chronic medications that would interfere with cognitive functioning such as medications for sleep, anxiety, narcotics for pain, use of illicit medical foods or cannabis.
4.2.3 Participants may not be receiving any other investigational agents.
4.2.4 History of allergic reactions attributed to compounds of similar chemical or biologic composition to oxaloacetate.
4.2.5 Uncontrolled intercurrent illness including, but not limited to, ongoing or active infection of Covid-19 or other virus, symptomatic congestive heart failure, unstable angina pectoris, cardiac arrhythmia, or psychiatric illness/social situations that would limit compliance with study requirements.
4.2.6 Pregnant or breast-feeding women are excluded from this study because the safety of oxaloacetate in this setting is unknown. A pregnancy test will be performed on all women with an intact uterus and ovaries who are not determined to be postmenopausal, as described in section 4.1.7.

The clinical trial provides an example on how to use the oxaloacetate agent to treat cognitive and muscular fatigue in patients with Post-COVID-19 fatigue, which is a novel use of oxaloacetate.

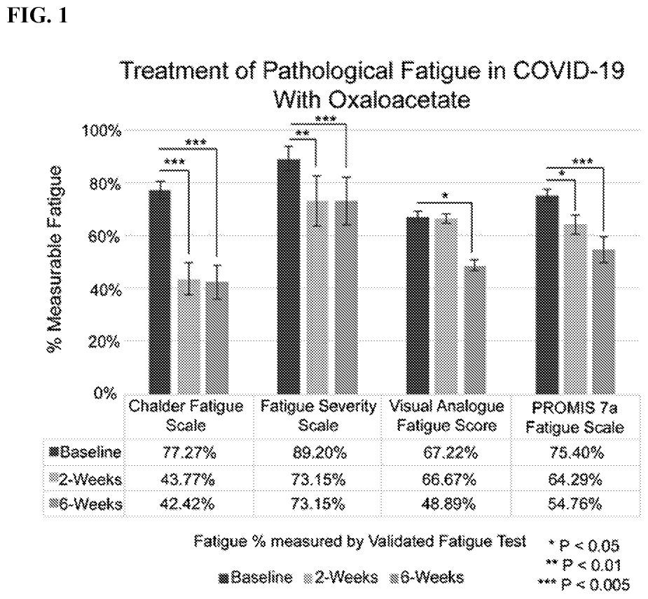

18 patients who suffered from “Long Haul” post-COVID-19 fatigue for an average of 6 months were recruited to a clinical trial. Each patient received oral 500 mg Oxaloacetate BID for 6 weeks. Fatigue was reduced by 45% from baseline at 6 weeks as measured by the Chalder Fatigue Scale, (P<0.005). Other fatigue scales corroborated this result. The Fatigue Severity Scale showed a 15% reduction in fatigue from baseline (P<0.005). The Visual Analogue Fatigue Scale showed a reduction of 56% (P<0.05). The PROMIS Fatigue Short Form 7a showed a 21% reduction in fatigue as compared to baseline (P<0.005). The results of the clinical trial data are shown in FIG. 1.

There were no significant adverse events during the trial. As these patients had fatigue for an average of 6 months, they meet the criteria for ME/CFS. The placebo effect for ME/CFS patients has been established as a very low effect, 5.9% as measured by the Chalder Fatigue Score using an oral medication. (Cho H J, Hotopf M, Wessely S. The placebo response in the treatment of chronic fatigue syndrome: a systematic review and meta-analysis. Psychosom Med. 2005; 67(2):301-13). The improvement in the Long-Haul COVID-19 patients with persistent fatigue was 7 times higher than could be expected with historical placebo. Therefore, oxaloacetate is shown to be effective in treating Long-Haul COVID-19 residual fatigue.

### Example 11

A Phase 1 trial evaluating the response of patients with Myasthenia Gravis fatigue treated by an oxaloacetate is planned. Fatigue will be assessed by the Chalder Fatigue Scale, the Fatigue Severity Scale, and PROMIS-Fatigue-Short Form 7a. Scores will be summarized as means and standard deviations and confidence intervals will be calculated. Changes will also be summarized as effect sizes.

The clinical trial provides an example on how to use the oxaloacetate agent to treat cognitive and muscular fatigue in patients with Myasthenia Gravis fatigue, which is a novel use of oxaloacetate.

### Example 12

A Phase 2 trial evaluating the response of patients with ME/CFS is in progress. Fatigue is assessed by the Chalder Fatigue Scale, the Fatigue Severity Scale, and PROMIS-Fatigue-Short Form 7a. Scores are summarized as means and standard deviations and confidence intervals will be calculated. Changes are also summarized as effect sizes. The study is being performed as a double-blind placebo-controlled study, wherein half of the patients were administered the active drug and the other half were administered placebo. The trial is being conducted in agreement with the International Conference on Harmonization (ICH) guidelines on Good Clinical Practice (GCP). All patients provide written informed consent to participate in the study prior to being screened.

Inclusion Criteria

The Fukuda criteria and the International Consensus Criteria (ICC) for diagnosis of ME/CFS were applied as inclusion criteria in this study.

The ICC for the diagnosis ME was presented in the Journal of Internal Medicine (International Consensus Criteria, ICC, Carruthers et al (2011) Volume 270, Issue 4 Pages 295-400) (49) and are an update of the previously used Fukuda (Fukuda et al (1994) Annals of Internal Medicine; 121:953-959) (50) and Canadian Criteria (Carruthers et al (2003) Journal of Chronic Fatigue Syndrome 11(1):7-115). (51)

Patient Population

Patients are to be recruited in the USA, initially a total of 80 women aged 18 to 70

Exclusion Criteria

Medication that is known/judged not to interfere with oxaloacetate will not be permitted. Medications which were not permitted were anti-epileptics or antipsychotics.

Patients with active substance abuse, pregnant women, women of childbearing age not on contraceptives and patients with abnormal laboratory parameters (e.g., Hb, white blood cells count, electrolytes, tests of liver and kidney functions, TSH, T4, B12, folic acid) judged to be of clinical significance will not be accepted.

Unstable therapies will not be allowed but stable therapies will be allowed. A stable therapy is defined as having started at least 6 months before the study and continued to be unchanged during the study period. Examples of such therapies are treatments with anti-depressants. Other stable therapies with hypnotics and anxiolytics were also allowed if they were given at doses recommended by the manufacturers.

Furthermore, analgesics such as NSAIDs, e.g., acetyl salicylic acid, paracetamol and duloxetine will be permitted as well as stable anti-hypertensive therapy. Acute or chronic medications for other medical conditions will be allowed based on clinical judgment.

Occasional use of over the counter (OTC) medications will be allowed at the investigator's discretion.

All concomitant medications, whether OTC or prescription, will be noted.

The clinical trial provides an example on how to use the oxaloacetate agent to treat cognitive and muscular fatigue in patients with Myalgic Encephalomyelitis/Chronic Fatigue Syndrome (ME/CFS) fatigue, which is a novel use of oxaloacetate.

69 patients with ME/CFS were enrolled in a clinical trial. 3 dosage levels of oral oxaloacetate were examined,
500 mg twice per day (BID) N=22, average time of disease 6.7 years, 68% women
1,000 mg twice per day (BID) N=25, average time of disease 5.7 years, 80% women
and 1,000 mg 3 times per day (TID) N=22, average time of disease 9.1 years, 86.4% women

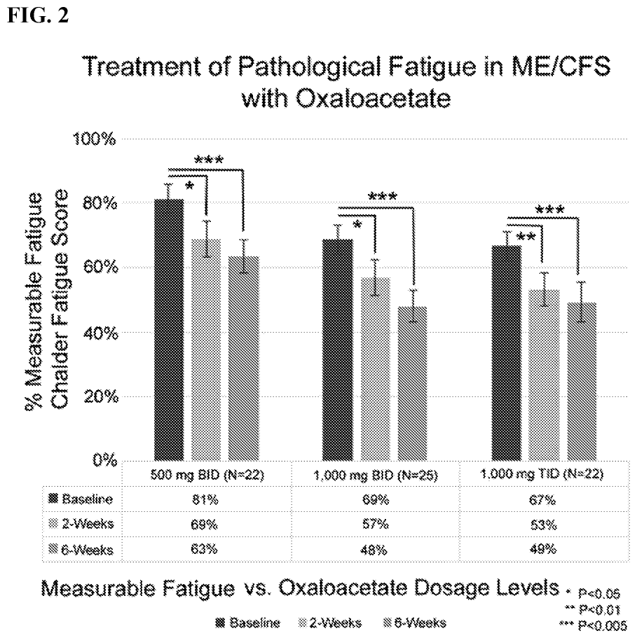

FIG. 2 graphically shows the results of measurable fatigue versus dosage levels and time.

All dosage levels showed significant reductions in fatigue at 2 weeks, as measured by the validated Chalder Fatigue Scale. Equally important, continued dosing with oxaloacetate further decreased fatigue. Fatigue reduction on average ranged from the low dose of 500 mg BID 15.3% reduction from baseline fatigue at 2 weeks to 17.5% reduction in fatigue in the high dose of 1,000 mg TID, indicating improvement with higher dosage. At 6-weeks, average improvement ranged from 21.9% reduction in fatigue at 500 mg BID to 30.2% reduction in fatigue at 1,000 mg BID.

There were no severe Adverse Effects recorded in the study. 4 patients experienced dyspepsia, which was resolved by taking the oxaloacetate with food. 1 patient experienced insomnia, which was resolved by moving the dosing schedule to breakfast and lunch rather than dinner.

Historical data in ME/CFS patients was used as a placebo comparison. (Cho H J, Hotopf M, Wessely S. The placebo response in the treatment of chronic fatigue syndrome: a systematic review and meta-analysis. Psychosom Med. 2005; 67(2):301-13). Fatigue treatment with an oral placebo in ME/CFS patients was measured with the Chalder Fatigue Scale and indicated the placebo effect to be 5.9%. Many other studies have indicated that the placebo effect in ME/CFS patients is very small. 76.8% of the patients in the study improved over historical placebo. As the average of all improvements seen with oxaloacetate ranged 3 times to 5 times as high as the historical placebo, oxaloacetate is shown to be effective in the treatment of pathological fatigue in ME/CFS.

A commercial “Medical Food” product providing 500 mg oxaloacetate capsules has been launched by the inventor based on the data from this study. More information at http://OxaloacetateCFS.com.

### Example 13

A cancer patient with recurrent stage 4 glioblastoma multiforme encountered significant fatigue as part of the ongoing disease. Although the cancer continued, fatigue associated with the cancer was ameliorated prior to the resolution of the disease with 6,000 mg of oxaloacetate taken daily. The patient continued this dosage for one year without any significant adverse events.

As pathological fatigue is common in cancer, it is an unexpected and novel use of oxaloacetate to ameliorate fatigue in a cancer patient.

### Example 14

A cancer patient with Stage 2 primary prostate cancer experienced significant fatigue. Upon dosing with 1,000 mg oxaloacetate daily, the pathological fatigue was ameliorated. The patient has been able to eliminate pathological fatigue for 4 years now and is continuing the dosage. No side effects have been noted at this dosage in this patient.

### Example 15

A cancer patient with stage 4 primary hepatocellular carcinoma experienced significant fatigue, and due to cancer progression, was placed in hospice. The patient began a dosage of 3,000 mg oxaloacetate per day and noticed a significant reduction in fatigue. The patient was able to continue this dosage for 10 months without side effects.

### Example 16

A cancer patient with stage 4 breast cancer experienced pathological fatigue. She began a dosage of 1,000 mg oxaloacetate per day and noticed a significant reduction in fatigue after 3 days. The patient was able to continue oxaloacetate dosage for 2 years.

### Example 17

A cancer patient with stage 3 colon cancer experienced pathological fatigue. He began a dosage of 1,000 mg oxaloacetate per day (500 mg BID) and noticed a significant reduction in fatigue. The patient continued to be fatigue-free and went into cancer remission, which was also fatigue free.

### Example 18

A patient with Alzheimer's disease experienced significant muscle and mental fatigue. A testimonial for the patient from the patient's caregiver/daughter/health professional indicates that oxaloacetate significantly reduces mental and physical pathological fatigue in the patient. The testimonial is shown below.

The above example shows efficacy in reducing pathological mental fatigue.

### Example 19

A patient diagnosed with Fibromyalgia had significant fatigue. Upon starting oxaloacetate, she noticed improvement with 2 capsules of 100 mg oxaloacetate per day. She increased her dosage to 6 capsules per day and has seen more than a 50% improvement in her fatigue.

What is claimed is:

1. A method of treating one or more symptoms of a disorder characterized by debilitating fatigue in a subject, the method comprising administering a therapeutic amount of an oxaloacetate compound to the subject; wherein said disorder is selected from the group consisting of post-COVID-19 fatigue, post-viral fatigue, myalgic encephalomyelitis/chronic fatigue syndrome, fibromyalgia, mental fatigue, post-stroke fatigue, Amyotrophic Lateral Sclerosis, Myasthenia Gravis, Huntington's disease, debilitating fatigue associated with Parkinson's disease, debilitating fatigue associated with Alzheimer's Disease, multiple sclerosis, narcolepsy, post cancer fatigue, fatigue associated with cancer with or without cytostatic treatment.

2. The method of claim 1, wherein said oxaloacetate compound is an anhydrous enol-oxaloacetate.

3. The method of claim 1, wherein said oxaloacetate is comprised from the group of enol-oxaloacetate, keto-oxaloacetate, hydrated oxaloacetate, or an oxaloacetate salt.

4. The method of any one of claims 1-3, wherein said disorder is selected from the group consisting of fibromyalgia, mental fatigue, Myalgic encephalomyelitis/chronic fatigue syndrome, fibromyalgia, Huntington's disease, post-COVID-19 fatigue, post-viral fatigue, Amyotrophic Lateral Sclerosis, Myasthenia Gravis, debilitating fatigue associated with Parkinson's disease, debilitating fatigue associated with Alzheimer's Disease, multiple sclerosis, and post-cancer fatigue.

5. The method of any one of claims 1-4, wherein said oxaloacetate agent is administered in a dose of about 100 to about 6,000 mg.

6. The method of any one of claims 1-5, wherein said oxaloacetate agent is administered in a dose of about 200 mg to about 3,000 mg.

7. The method of any one of claims 1-6, wherein said oxaloacetate agent is administered once, twice or three times a day.

8. The method of any one of claims 1-7, wherein the oxaloaacetat compound is in a pharmaceutical composition.

9. A method of treating one or more symptoms of a disorder characterized by debilitating fatigue in a subject, the methods comprising administering a therapeutic amount of a compound to reverse metabolic dysfunction to the subject; wherein said dysfunction is selected from the group consisting of increased glycolysis, chronic activation of NF-kB, decreased NAD+/NADH ratio, mitochondrial insufficiency, reduced activation of AMPK, and combinations thereof.

10. The method of claim 9 wherein said compound is an oxaloacetate compound.

11. The method of claim 10, wherein said oxaloacetate compound is selected from the group consisting of enol-oxaloacetate, keto-oxaloacetate, hydrated oxaloacetate and an oxaloacetate salt.

12. The method of claim 10 or 11, wherein said oxaloacetate compound is anhydrous enol oxaloacetate

13. The method of any one of claims 9-12, wherein said disorder is selected from the group consisting of fibromyalgia, mental fatigue, Myalgic encephalomyelitis/chronic fatigue syndrome, fibromyalgia, Huntington's disease, post-COVID-19 fatigue, post-viral fatigue, Amyotrophic Lateral Sclerosis, Myasthenia Gravis, debilitating fatigue associated with Parkinson's disease, debilitating fatigue associated with Alzheimer's Disease, multiple sclerosis, and post-cancer fatigue.

14. The method of any one of claims 10-13, wherein said oxaloacetate compound is administered in a dose of about 100 to about 6,000 mg.

15. The method of any one of claims 10-14, wherein said oxaloacetate compound is administered in a dose of about 200 mg to about 3,000 mg.

16. The method of any one of claims 10-15, wherein said oxaloacetate compound is administered once, twice or three times a day.

17. The method of any one of claims 9-16, wherein the compound to reverse metabolic dysfunction is in a pharmaceutical composition.
